(19) United States
(12) Patent Application Publication
(10) Pub. No.: US 2026/0183419 A1
(43) Pub. Date: Jul. 2, 2026

(54) COMPOSITIONS AND ARTICLES COMPRISING (NANO)DIAMOND PARTICLES

(71) Applicant: Debina Diagnostics, Inc., Newtown Square, PA (US)

(72) Inventors: Giora Z. Feuerstein, Bryn Mawr, PA (US); Mark E. Sternberg, Bryn Mawr, PA (US)

(73) Assignee: Debina Diagnostics, Inc., Newtown Square, PA (US)

(21) Appl. No.: 19/284,596

(22) Filed: Jul. 29, 2025

Related U.S. Application Data
(63) Continuation of application No. 17/751,924, filed on May 24, 2022, now abandoned.
Application No. 17/751,924 is a continuation-in-part of application No. 17/620,064, now abandoned.
(60) Provisional application No. 62/862,802, filed on Jun. 18, 2019.

(51) Int. Cl. A61K 47/69; A61K 9/00; A61K 31/337; A61K 31/395; A61K 31/475; A61K 31/513; A61K 31/519; A61K 31/655; A61K 31/675; A61K 31/704; A61K 31/7048; A61K 31/7068; A61K 49/00; A61P 35/00; C01B 32/28; C09K 11/02; C09K 11/65

(52) U.S. Cl. CPC A61K 47/6923; A61K 9/0019; A61K 31/337; A61K 31/475; A61K 31/513; A61K 31/519; A61K 31/5545; A61K 31/655; A61K 31/675; A61K 31/704; A61K 31/7048; A61K 31/7068; A61K 49/0067; A61P 35/00; C01B 32/28; C09K 11/025; C09K 11/65; C01P 2004/61; C01P 2004/62

(57) ABSTRACT

Compositions and articles comprising diamond particles, such as nanodiamond based pharmaceutical compositions, are generally provided. In some embodiments, the articles and methods comprising (nano)diamond particles may be useful for monitoring and/or treating a disease (e.g., in a subject).

### RELATED APPLICATIONS

This application is a continuation-in-part of co-pending U.S. Non-Provisional application Ser. No. 17/620,064, filed Dec. 16, 2021, which is a national stage filing under 35 U.S.C. § 371 of international application number PCT/US2020/038452, filed Jun. 18, 2020, which claims priority under 35 U.S.C. § 119(e) to U.S. Provisional Application No. 62/862,802, filed Jun. 18, 2019, and entitled “COMPOSITIONS AND ARTICLES COMPRISING NANODIAMOND PARTICLES,” each of which is incorporated herein by reference in its entirety for all purposes.

### TECHNICAL FIELD

Compositions and articles comprising diamond particles, such as nanodiamond based pharmaceutical compositions, are generally provided.

### BACKGROUND

The use of nanomaterials for novel diagnostics and therapeutic purposes is a fast progressing scientific discipline that builds on the bioengineering of biological and pharmaceutical entities in combinations with physical materials.

However, improved articles and methods are needed.

### SUMMARY

Diamond particles and related devices and methods, such as nanodiamond particles (e.g., fluorescent nanodiamond particles) for administration of a therapeutic agent to a subject and/or monitoring the progression of a disease within a subject.

The subject matter of the present invention involves, in some cases, interrelated products, alternative solutions to a particular problem, and/or a plurality of different uses of one or more systems and/or articles.

In one aspect, articles (e.g., configured for administration of a therapeutic agent, for use with a subject) are provided. In some embodiments, the article comprises a plurality of fluorescent diamond particles and a therapeutic agent bound to at least a portion of the fluorescent diamond particles, wherein the article is configured for prolonged residence internal to an organ of a subject.

In some embodiments, the article comprises an injection component configured to administer a composition to the subject and a reservoir associated with the injection component containing the composition, the composition comprising a plurality of fluorescent diamond particles.

In another aspect, pharmaceutical compositions are provided. In some embodiments, the pharmaceutical composition comprises an intravenous carrier fluid and a plurality of fluorescent diamond particles suspended in the intravenous carrier fluid.

In yet another aspect, methods (e.g., of treating a disease, of monitoring disease progression in a subject suspected of having a disease) are provided. In some embodiments, the method comprises administering intravenously, to a subject, a plurality of diamond particles and a therapeutic agent bound to at least a portion of the diamond particles, wherein the plurality of diamond particles is configured for prolonged residence internal to an organ of a subject.

In some embodiments, the method comprises administering to the subject a plurality of diamond particles, after the step of administering, obtaining a first image of a location internal to the subject suspected of containing the plurality of diamond particles, obtaining, after a predetermined period of time, a second image of the location internal to the subject suspected of containing the plurality of diamond particles, and measuring a morphological change of the location internal to the subject, between the first image and the second image, relative to the plurality of diamond particles, wherein the morphological change is associated with progression of the disease.

In some embodiments, use of a plurality of fluorescent diamond particles in the manufacture of a medicament for the treatment of liver disease and/or liver cancer, are provided. In some embodiments, use of a plurality of fluorescent diamond particles in the manufacture of a medicament for monitoring of disease progression, are provided.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic illustration of a system including fluorescent nanodiamond particles, according to one set of embodiments;

FIG. 1B is a schematic presentation of method used for quantification of FNDP-(NV) uptake into cells, according to one set of embodiments;

FIGS. 2A-2B show fluorescence microscope images of paraffin sections (5 μm) of liver obtained from rats treated or not with FNDP-(NV)˜700/800 nm (FNDP-(NV)), according to one set of embodiments. In FIG. 2A, images of tissue sections analyzed with 10× objective with 1.6× extension are shown and in FIG. 2B images of tissue sections analyzed with oil 40× objective with the left images showing overlapped three colors red (FNDP-(NV)), blue (DAPI-nuclei), Green (phalloidin-cytoskeleton) shown with different shades of grey while images on the right show overlapped two colors red (FNDP-(NV)), blue (DAPI-nuclei) also shown with different shades of gray and the upper images in each panel represent FNDP-(NV)-treated rats, lower images in each panel control (PBS-treated rats) and areas occupied by the particles are indicated by white arrows, according to some embodiments;

FIGS. 3A-3H show “panoramic” images of hepatic lobes demonstrate intra-lobule heterogeneity of particles distribution, according to one set of embodiments, FIG. 3A and (FIG. 3B depict total panoramic view of a sagittal section from representative hepatic lobes from two animals with these figures constructed by ‘stitching’ 4× images using FSX100 microscope with the Phalloidin stained sections (5 μm) imaged in the green channel (show in a shade of grey), and presence of FNDP-(NV) imaged in the red (shown in grey) channel; particles in the image have been magnified by thresholding and repeated dilations for visualization at very low resolution; hexagons are over-laid in the figure to indicate example hepatic lobules with areas indicated in gold are magnified in other panels and (FIG. 3C) presents four hepatic lobules demonstrating preferential particle distribution at the boundaries of the ‘hexagonal’ lobules format and (FIG. 3D) present 10× image of a single hepatic lobule showing preferential FNDP-(NV) deposition; large FNDP-(NV) aggregates are seen distributed non-uniformly with hepatic lobule indicated by dashed hexagon and (FIG. 3E), (FIG. 3F) present 10× image of a single hepatic lobule after thresholding and dilating to improve visibility of very small aggregates, to demonstrate zonal deposition and (FIG. 3G), (FIG. 3H) providing magnified images of areas of vasculature from panel (FIG. 3A) indicated by gold dashed square and (FIG. 3I) as a schematic illustration of hepatic lobule that demarcates the various metabolic zones, according to some embodiments;

FIGS. 4A-4B show mathematical plots of size distribution of FNDP-(NV) aggregates in liver lobules; figures of one entirely liver lobule from two animals were stitched from 10× images on an FSX100 microscope; Maximum Entropy criteria was used to threshold stitched figures in ImageJ and the resulting detected FNDP-(NV) assemblies were sized and counted; (FIG. 4A) Distribution of FNDP-(NV) assembly sizes. (FIG. 4B) Distribution of total particle mass estimated by the area of each assembly, according to some embodiments;

FIGS. 5A-5D show laser scanning confocal microscope images of liver sections (50 μm) obtained from rats treated with FNDP-(NV), according to one set of embodiments. (FIG. 5A) Parenchymal area of liver with indicated cells in yellow circles with up-taken particles. Inserts on the bottom and on the right of the photo represent vertical projection of images performed along the yellow lines. Yellow arrows indicate location of particles. (FIG. 5B) Parenchymal area of liver where yellow circles suggest aggregates of particles within liver sinusoids/venues. Inserts on the bottom and on the right represent vertical projection of images performed along the yellow lines. Yellow arrows indicate particles localized in sinusoids/venules. (FIG. 5C) Area of abundantly vascularized segment of the hepatic lobule where white circles particles suggest sub-Substitute endothelial and adventitial location of particles. Parenchymal cells with supposedly internalized particles are indicated in yellow circles. Inserts on the bottom and on the right represent vertical projection of images performed along the yellow lines. Yellow arrows indicate particles internalized in parenchymal cells. (FIG. 5D) Area of the liver hilum where white circles indicate particles associated with adventitial cellular elements. Inserts on the bottom and on the right represent vertical projection of images performed along the yellow lines. Yellow arrows indicate internalized particles into the vascular cells;

FIG. 6 shows confocal 3D reconstruction of hepatocytes with differing amount of incorporated FNDP-(NV), according to one set of embodiments. Confocal image stacks from 50 μm sections stained with DAPI (blue) and phalloidin (green) with incorporated nanodiamonds (red) shown in different shades of grey with image stacks were taken on a Fluoview F1000 confocal microscope and reconstructed using volume viewer in ImageJ. Particles inclusions within these cells (indicated by yellow arrow) include both sparse and dense FNDP-(NV) collections internalized in the cells. Left panel represents vehicle control. Middle panel represent low load particle and right panel represent high load particle in 2 separate cells.

FIGS. 7A-7D show plots related to internalization of different concentrations of FNDP-(NV) into HepG-2 and HUVEC cells over time, according to one set of embodiments. (FIG. 7A), (FIG. 7B), (FIG. 7C) depict dose and time dependent uptake of FNDP by HepG-2 cells and HUVEC exposed to various concentrations of FNDP-(NV). Exponential curves were fitted for all three doses (high-dose 0.1 mg/ml; medium-dose 0.05 mg/ml, low-dose 0.025 mg/ml) of particles. (FIG. 7D) Total uptake of FNDP after 20 hours by HepG-2 cells and HUVEC exposed to various concentrations of FNDP. Error bars for all panels represent SD from quadruplicated samples. (*) P<0.001 compared to 0.025 mg/ml by two-tailed Student's test; (†) P<0.001 compared to 0.05 mg/ml by two-tailed Student's test;

FIGS. 8A-8B show fluorescence microscope of images of HepG-2 cell and HUVEC obtained after 2 and 20 hours incubation with FNDP-(NV), according to one set of embodiments. Images of HepG-2 cells (FIG. 8A) and HUVEC (FIG. 8B) obtained from fluorescence microscope analysis using 160× and 400× magnification after 2 or 20 hours of exposure to FNDP-(NV). Images of 160× magnification are presented in overlapped three colors fluorescence (green-FITC-phalloidin, red-FNDP-(NV), blue DAPI) shown in different shades of grey with images of 400× magnification are presented in overlapped three colors fluorescence (green, red, blue) (left panels), and two colors fluorescence (red and blue) (right panels). White arrows denote example of the cytoplasmic phase of particles transition; Grey arrows indicated peri-nuclear assembly of large number of particle;

FIG. 9 shows representative images demonstrating various stages of HUVEC division in the presence of FNDP-(NV), according to one set of embodiments. HUVEC were treated with 0.05 mg/ml of FNDP-(NV) for 20 hours. Images of 400× or 640× magnifications are presented in overlapped three colors fluorescence (green-FITC-phalloidin, red-FNDP-(NV), blue-DAPI). Titles of the various phases noted are visual images of predicted cell replication mechanism;

FIGS. 10A-10B show the effect of passive adsorption of BSA on aggregation and surface potential of FDP-NV functionalized with carboxyl groups and suspended in water, culture medium and biological buffers where the particles were suspended in the various dispersants, applied into capillary cuvettes, and positioned into a Zetasizer instrument (Malvern Inc.) for measurement Z-average, diameter size (FIG. 10A) and potential (FIG. 10B) and where error bars represent SD from three measurements of independent samples. (*) P<0.01 and (**) P<0.001 for difference between FDP-NV-BSA and native FDP-NV, in particular dispersant, calculated using One Way ANOVA., according to one set of embodiments;

FIGS. 11A-11D show effect of FDP-NV on cell proliferation determined by evaluation of direct cell number, where the graphic presentation of numbers of HepG-2 cells (FIG. 11A) and HUVEC (FIG. 11B) obtained after incubation or not with FDP-NV-BSA, or vincristine and where error bars represent SD from 5 independent wells, and application for 7 observation fields for each well. (*) P<0.001 between control and treated group calculated using One Way ANOVA, and where representative images of observation fields of HepG-2 cells (FIG. 11C) or HUVEC (FIG. 11D) applied for determination of cell numbers using ImageJ software with images that were obtained using fluorescence microscope (Olympus IX81) with application 10× objective and DAPI (blue) and TRITC (red) filters (shown in different shades of grey) with white arrows indicating internalized particles into flanking cells of HepG-2 colonies, according to some embodiments;

FIGS. 12A-12B show the effect of FNDP-(NV) on HepG-2 (FIG. 12A) and HUVEC (FIG. 12B) Redox state tested in MTT assay where error bars represent one SD from three independent experiments with One-way ANOVA calculated between control and compound treated group, (*) P<0.01 and (**) P<0.001, according to some embodiments;

FIGS. 13A-13B shows the effect of FNDP-(NV) on HepG-2 (FIG. 13A) and HUVEC (FIG. 13B) esterase activity monitored calcein AM assay where the graphic presentation of conversion of calcein AM to green-fluoresce calcein by esterases present in live HepG-2 cells (FIG. 13A) and HUVEC (FIG. 13B) is shown with error bars representing SD from three independent experiments and where One-way ANOVA was calculated between control and compound treated group, (*) P<0.01 and (**) P<0.001, according to some embodiments;

FIG. 14A shows the effect of FNDP-(NV) on migration of HUVEC stimulated by 2% FBS in scratch assay showing scratch closure” stimulated by 2% FBS in the presence or absence of FNDP-NV-BSA with non-stimulated cells (negative control) treated with a medium containing 0.1% FBS and error bars representing SD from three independent experiments, (*) P<0.001 for comparison with control (2% FBS treated) in One-way ANOVA, according to one set of embodiments;

FIG. 14B shows the effect of FNDP-(NV) on migration of HUVEC stimulated by 2% FBS in scratch assay with images of scratches obtained using fluorescence microscope (Olympus IX81) with application 20× magnification and DAPI (blue) and TRITC (red) filters shown in different shades of grey, according to one set of embodiments;

FIGS. 15A-15B show the effect of FDP-NV on phosphorylation of MAPK Erk1/2 induced by FBS with 24 hour serum-starved HepG-2 cells (FIG. 15A) or HUVEC (FIG. 15B) stimulated with 2% FBS by 10 and 20 minutes and total MAPK Erk1/2 re-probed in PVDF membrane after stripping anti-phospho antibody with right plot bars presenting a ratio of intensity of total protein bands to phosphorylated protein bands and green bars presenting ratios for control (non-treated cells), whereas red bars for FDP-NV treated cells and left panes showing representative blot images for each cell type with error bars representing SD for three independent experiments, (*) P<0.01 for comparison between treated or non-treated cells with FDP-NV-BSA 0.1 mg/ml by ‘One-way ANOVA’, according to one set of embodiments;

FIGS. 16A-16B shows the identification of phospho-and total-MAPK Erk1/2 in cytoplasm and nuclei of HepG-2 cells and HUVEC in the presence and absence of FDP-NV and TPA, HepG-2 cells (FIG. 16A) or HUVEC (FIG. 16B) were treated or not with FDP-NV-BSA (0.1 mg/ml), and after 24 hour serum-starvation, stimulated or not with TPA with cells lysed and fractionated into cytoplasmic and nuclear fractions and fractions that are subjected to WB using indicated antibodies; Mek-1 was used as marker for cytoplasm fraction, whereas HDAC1 as nucleus fraction;

FIGS. 16C-16D shows the identification of phospho-and total-MAPK Erk1/2 in cytoplasm and nuclei of HepG-2 cells and HUVEC in the presence and absence of FDP-NV and TPA with HepG-2 cells (C) or HUVEC (D) grown on chamber slides and serum-starved for 24 hours, following exposed to FDP-NV-BSA. After treatment or not with TPA, cells were immune-stained with anti-phospho-MAPK Erk1/2, following with goat anti-rabbit tagged with FITC. slides analyzed under fluorescence microscope (Olympus IX81) using 400× magnification with oil objective and FITC (green) and TRITC (red) filters with overlapped areas of green and red in yellow shown with various shades of grey and with white arrows indicating high accumulation of particles in TPA-treated cells if compare with non-treated cells and blue arrows indicate nuclei of cell non-treated with TPA with red arrows indicating nuclei of cell treated with TPA, according to one set of embodiments;

FIG. 17A shows the effect of FDP-NV on induction of apoptosis and ER stress in HepG-2 cells and HUVEC with a Western blot analysis of cleavage of caspase 3 in the presence or absence of FDP-NV (0.1 mg/ml) in HepG-2 and HUVEC. Vincristine was used as positive control for apoptosis. Localization of molecular weight markers is indicated by arrows on the left side of images, according to some embodiments; and

FIG. 17B shows the effect of FDP-NV on induction of apoptosis and ER stress in HepG-2 cells and HUVEC with a Western blot analysis of expression of chaperons in ER in the presence or absence of FDP-NV (0.1 mg/ml) in HepG-2 cells and HUVEC. Tunicamycin was used as positive control for ER-stress, according to some embodiments.

FIG. 18A shows an image of FDP-DOX suspended in PBS (pH=7.4) after vortex, observed under fluorescence microscope, according to some embodiments. The max diameters of the 6 largest agglomerates are as followed: 1) 6.4 μm, 2) 5.8 μm, 3) 4.6 μm, 4) 5.4 μm, 5) 5.1 μm, 6) 3.7 μm.

FIG. 18B shows an image of FDP-DOX after sonication, observed under fluorescence microscope, according to some embodiments. The max diameters of the 6 largest particles are as followed: 1) 1.9 μm, 2) 1.7 μm, 3) 1.8 μm, 4) 1.2 μm, 5) 1.2 μm, 6) 1.6 μm. FDP-DOX were suspended in PBS (0.1 mg/ml) and subjected to sonication over 10 min. period. Few drops of suspensions were applied on microscope glass slides and inspected by fluorescence microscope (Olympus IX81) with TRITC (red) filter and 40× oil objective. White arrows indicate measured particles. The measurement of particles'diameters was performed using ImageJ software.

FIG. 18C shows an image of FDP-DOX suspended in PBS (pH=7.4) after vortex, observed under light microscope, according to some embodiments.

FIG. 18D shows an image of FDP-DOX after sonication, observed under light microscope, according to some embodiments. FDP-DOX were suspended in PBS, applied on the hemocytometer scale, and analyse under light microscope. Abbreviations: FDP-DOX, fluorescence diamond particles with NV active center and size of diameter 800 nm containing adsorbed doxorubicin; TRITC, tetramethyl rhodamine; PBS, phosphate buffered saline pH=7.4.

FIG. 19A shows the accumulation of FDP-NV-700/800 nm in the liver after single and double doses administration was determined by NIR fluorescence measured using IVIS in isolated organs, according to some embodiments. Mice were IV injected with a single dose of 774 μg of FDP-NV-700/800 nm or double dose of 774 μg of FDP-NV-800 nm (1,548 μg of total administrated amount) and organs were collected on post-injection day 5. IVIS images on the right present liver from double dose. Error bars represent SD for five animals per single dose group (N=5) and three animals per double dose group (N=3). (*) P<0.001 and (**) P<0.01 calculated using One-way ANOVA.

FIG. 19B, according to some embodiments shows the distribution of FDP-NV-700/800 nm among the indicated organs after single and double dose administration determined by NIR fluorescence measured by IVIS in isolated organs. Mice were IV injected with a single dose of 774 μg of FDP-NV-700/800 nm or double dose of 774 μg of FDP-NV-700/800 nm (1,548 μg of total administrated amount) and organs were collected on post-injection day 5. Error bars represent SD for five animals per single dose group (N=5) and three animals per double dose group (N=3). (*) P<0.01 calculated using One-way ANOVA. Images above bar graphs present organs treated (bottom rows) or not treated (upper rows) with FDP-NV-700/800 nm.

FIG. 19C shows the effect of FDP-NV-700/800 nm on the liver parameters in collected blood, according to some embodiments. Mice were IV injected with a single dose of 774 μg of FDP-NV-700/800 nm and blood was collected on post-injection day 5. Error bars represent SD for three animals per group (N=3).

FIG. 19D shows the effect of FDP-NV-700/800 nm on the selected blood parameters, according to some embodiments. Mice were IV injected with a double dose of 774 μg of FDP-NV-700/800 nm (1,548 μg of total administrated amount) and blood was collected on post-injection day 5. Error bars represent SD for four animals per control group (N=4), and five animals for particles treated group (N=5). (*) P<0.01 calculated using One-way ANOVA. For the platelet count, the FDP-NV-700/800 nm treated animals was decreased by 27.6%. Abbreviations: FDP-NV-700/800 nm, fluorescence diamond particles with NV active center and size of diameter 700-800 nm; IVIS, In Vivo Imaging System; SD, standard deviation; S, single dose; D, double dose; N, number of animals per group; NIR, near infra-red; ALT, alanine transaminase; AST, aspartate aminotransferase; ALB, albumin; ALP, alkaline phosphatase; TP, total protein; TBIL, total bilirubin; RBC, red blood cells; WBC, white blood cells; PLT, platelets; HTC, hematocrit; HGB, hemoglobin.

FIG. 20A shows dose-dependent accumulation of FDP-DOX in mouse liver, according to some embodiments. Animals were IV injected with the different doses of particles (indicated) and organs were isolated at day 5. NIR fluorescence of isolated livers were measured using IVIS. Error bars represent SD for variable number animals per injected dose (N=2 to 10 animals per group). (*) P<0.001 calculated using One-way ANOVA.

FIG. 20B shows linear dose-dependent accumulation of FDP-DOX in mouse liver, according to some embodiments. Animals were IV injected with the different doses of particles (indicated) and organs were isolated at day 5. NIR fluorescence of isolated livers were measured using IVIS. Error bars represent SD for variable number animals per injected dose (N=2 to 10 animals per group). Liner evaluation was prepared for four parameters logistic curve using SigmaPlot software. (*) P<0.001 calculated using one-way ANOVA.

FIG. 20C shows the effect of FDP-DOX on the liver parameters is collected blood, according to some embodiments. Mice were IV injected with total 1.2 mg dose of FDP-DOX and blood was collected on post-injection day 5. Error bars represent SD for three animals per particles treated group (N=3) and nine animals for control group (N=9). (*) P<0.05 calculated using One-way ANOVA.

FIG. 20D shows the effect of FDP-DOX on the selected blood parameters, according to some embodiments. Mice were IV injected by double dose of 2400 μg of FDP-DOX per mouse and blood was collected on post-injection day 5. Error bars represent SD for four animals per control group (N=4), and three animals for particles treated group (N=3). (*) P<0.001 calculated using one-way ANOVA. Abbreviations: FDP-DOX, fluorescence diamond particles with NV active center and size of diameter 800 nm containing adsorbed doxorubicin; SD, standard deviation; N, number of animals per group; NIR, near infra-red; ALT, alanine transaminase; AST, aspartate aminotransferase; ALB, albumin; ALP, alkaline phosphatase; TP, total protein; TBIL, total bilirubin; RBC, red blood cells; WBC, white blood cells; PLT, platelets; HTC, hematocrit; HGB, hemoglobin.

FIG. 21 shows scanning electron microscope (SEM) images of the liver of mice treated with FDP-DOX, according to some embodiments. Surface of slices of fixed liver tissue was analyzed using SEM under different magnifications (indicated above each image). Framed areas on lower magnification (yellow dashed lines) indicate images taken in higher magnifications. Diameters of FDP-DOX clusters are marked by red dashed arrows. Diameters of selected single FDP-DOX are indicated by red arrows. White arrow indicates the central vein.

FIG. 22A shows images of whole bodies fluorescence and bioluminescence, according to some embodiments.

FIG. 22B shows images and pictures of livers with tumors measured by a proxy biomarker (fluorescence and bioluminescence), according to some embodiments.

FIG. 22C shows images of separated tumors fluorescence and bioluminescence, according to some embodiments.

FIG. 23A shows body weights of mice were measured on the beginning of experiment (day 0) and end point of experiment with sacrificed animals (day 66), according to some embodiments. Bars present values of weights of control animals with developing tumor (N=2) and mice with developing tumor treated with FDP-DOX (N=5).

FIG. 23B shows liver function parameters were measured in the blood using standard procedures, according to some embodiments. Error bars present SD for control mice which were not subjected for tumor inoculation (N=10; data obtained from vendor), and mice with developed tumor and treated with FDP-DOX (N=5). The values for control mice with developed tumor are presented as a mean from two animas (N=2)

FIG. 23C shows blood parameters were measured in the blood using standard procedures, according to some embodiment. Error bars present SD for control mice which were not subjected for tumor inoculation (N=10; data obtained from vendor), and mice with developed tumor and treated with FDP-DOX (N=5). The values for control mice with developed tumor are presented as a mean from two animals (N=2). Abbreviations: FDP-DOX, fluorescence diamond particles with NV active center and size of diameter 800 nm containing adsorbed doxorubicin; N, number of animals per group; NIR, near infra-red; ALT, alanine transaminase; AST, aspartate aminotransferase; ALB, albumin; ALP, alkaline phosphatase; TP, total protein; TBIL, total bilirubin; RBC, red blood cells; WBC, white blood cells; PLT, platelets; HTC, hematocrit; HGB, hemoglobins.

FIG. 24 shows representative images of paraffin sections of tumor and normal liver of mice treated or not with FDP-DOX. Note: Paraffin sections were stained with AFP and H&E for colorimetric imaging, and green fluorescence AFP and DAPI for fluorescence imaging. Fluorescence FDP-DOX are shown in red. Borders between tumor and normal liver tissue are shown by yellow or pink dashed lines. Images a, b, k show immunohistochemistry staining with anti-AFP observed under different magnification; images c, d, i show H&E staining observed under different magnifications; fluorescence images g, h, m, o show presence of FDP-DOX in red observed under different magnifications; fluorescence images i, j, p show presence of AFP (green) and nuclei (blue) under different magnifications; fluorescence image n shows combined colors for AFP (green), FDP-DOX (red) and nuclei (blue). Abbreviations: FDP-DOX, fluorescence diamond particles with NV active center and size of diameter 800 nm containing adsorbed doxorubicin; DAPI, 4′,6-diamidino-2-phenylindole; H&E, hematoxylin, and eosin according to some embodiments.

FIG. 25A shows the progression of bioluminescence measured by whole mouse body scanning using IVIS. Error bars represent SD from four animas per control group (N=4) and eight animals for tumor bearing animals (N=8). (*) P<0.001 calculated using One-way ANOVA according to some embodiments.

FIG. 25B shows bioluminescence images of entire bodies of mice in different time points according to some embodiments.

FIG. 25C shows bioluminescence images and photos of isolated livers from control and tumor bearing mice. Areas of liver affected by tumor are framed in the photos by red according to some embodiments.

FIG. 25D shows liver function parameters measured in the control and tumor bearing mice on day 7 and 14. Error bars represent SD from four animas per control group (N=4) and eight animals for tumor bearing animals (N=8). Abbreviations: luc, luciferase; SD, standard deviation; N, number of animals per group according to some embodiments.

FIG. 26A shows progression of bioluminescence measured by whole mouse body scanning using IVIS. Error bars represent SEM from five animals (N=4). (*) P<0.01, (**) P<0.05) calculated using One-Way ANOVA according to some embodiments.

FIG. 26B shows progression of bioluminescence measured by whole mouse body scanning using IVIS. Error bars represent SD from five animals (N=5) according to some embodiments.

FIG. 26C shows images of bioluminescence of entire bodies of mice in different time points according to some embodiments.

FIG. 26D shows images of bioluminescence of isolated livers containing or not developed tumors in different time points according to some embodiments.

FIG. 26E shows photos of isolated livers containing or not (control) developed tumor. Areas of liver affected by tumor are framed by red according to some embodiments.

FIG. 26F shows bioluminescence images of non-liver organs from control and tumor bearing mice isolated on day 28. Abbreviations: luc, luciferase; SEM, standard error of the mean; N, number of animals per group, according to some embodiments.

FIG. 27A shows that blood was collected from the animals on the indicated time points, and liver function parameters were analyzed. Error bars represent SD from five animals per group (N=5). (*) P<0.01, (**) P<0.05) calculated using One-Way ANOVA for comparison with control, according to some embodiments.

FIG. 27B shows progression weight of whole body and isolated livers with tumor on the indicated time points. Abbreviations: luc, luciferase; SD, standard deviation; N, number of animals per group; NIR, near infra-red; ALT, alanine transaminase; AST, aspartate aminotransferase; ALB, albumin; ALP, alkaline phosphatase; TP, total protein; TBIL, total bilirubin, according to some embodiments.

FIG. 28A shows weight and fluorescence of entire bodies of mice. Error bars represent SD from three animals per group (N=3), according to some embodiments.

FIG. 28B shows weight and fluorescence of all liver and separated lobs of liver from mice. Separated lobes were marked as follow: (1) median lobe, (2) left lobe, (3) right lobe, (4) caudate lobe. Error bars represent SD from three animals per group, according to some embodiments.

FIG. 28C shows weight and fluorescence of organs separated from mice. Error bars represent SD from three (FDP-DOX-34) and two (vehicle) animals per group. Total fluorescence for 1 gram of the spleen tissue pf animals with injected FDP-DOX-34 is 5.4×10¹⁰±0.4×10¹⁰ (SD from three animals per group). Abbreviations: FDP-DOX, fluorescence diamond particles with NV active center and size of diameter 800 nm containing adsorbed doxorubicin (34 μg/mg); standard deviation; N, number of animals per group, according to some embodiments.

### DETAILED DESCRIPTION

Compositions and articles comprising diamond particles, such as diamond based pharmaceutical compositions, are generally provided. In some embodiments, the articles and methods comprising diamond particles may be useful for monitoring and/or treating a disease (e.g., in a subject). In some embodiments, an article may be configured to administer a plurality of diamond particles (e.g., fluorescent (nano)diamond particles) that can be used to deliver a therapeutic agent bound to the (nano)diamond particles. For example, the plurality of (nano)diamond particles may be administered to a subject such that at least a portion of the plurality of (nano)diamond particles reside at a location internal to the subject (e.g., within an organ such as the liver). In some embodiments, the (nano)diamond particles may be used as a diagnostic tool. For example, in some embodiments, a plurality of (nano)diamond particles may be administered (e.g., via intravenous injection) to a subject. In some such embodiments, an image of the location suspected of containing the plurality of (nano)diamond particles may be obtained, and, after a diagnostically relevant period of time, a second image of the same location internal to the subject suspected of containing the plurality of (nano)diamond particles may be obtained. In some embodiments, the first image and/or the second image is based on near infrared and/or fluorescent emissions (e.g., by the (nano)diamond particles). In some embodiments, a comparison of the first image and the second image may provide diagnostic information including, for example, progression of a disease state (e.g., cancer). For example, areas in the second image which comprise new tissue without the plurality of (nano)diamond particles may, in some cases, indicate malignant growth. As such, (nano)diamond particles, in some embodiments, may be useful for monitoring the progression of a disease. In some embodiments, the first image and the second image are obtained under similar (e.g., identical) conditions (e.g., same wavelength of excitation and/or emission).

A “subject”, as used herein, refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. The embodiments described herein may be, in some cases, directed toward use with humans. The embodiments described herein may be, in some cases, directed toward veterinary use. In some embodiments, a subject may demonstrate health benefits, e.g., upon administration of the (nano)diamond particles.

In some embodiments, (nano)diamond particles described herein may be configured for prolonged residence time within one or more organs (e.g., the liver) of a subject. For example, the progression of tumor growth may be monitored by administering a plurality of (nano)diamond particles to a subject and imaging organs suspected of tumor growth as described above.

Non-limiting examples of suitable target organs and/or tissues (e.g., for prolonged residence time, for treatment, for diagnostics, etc.) for the (nano)diamond particles described herein include liver, spleen, pancreas, intestines, stomach, lung, kidney, spleen, breast, heart and brain.

In some embodiments, (nano)diamond particles described herein may be configured to deliver a therapeutic agent (e.g., to an organ internal to a subject, to a surface of the subject's body such as the skin). In some embodiments, a therapeutic agent may be bound, at least partially, to a plurality of (nano)diamond particles. In some cases, the (nano)diamond particle bound to the therapeutic agent may be administered to a subject (e.g., to provide a therapeutic effect).

Because (nano)diamond particles may be configured to have a relatively prolonged residence internal to a location internal to the subject (e.g., an organ), therapeutic agents delivered using (nano)diamond particles may advantageously deliver a therapeutic agent over a prolonged period of time. In some embodiments, (nano)diamond particles are configured for prolonged residence in a subject or internal to an organ of a subject. In some embodiments, the (nano)diamond particles are configured for residence (e.g., have a size and/or shape that facilitates residence). In some embodiments, the (nano)diamond particles are configured for residence in an organ for greater than or equal to 1 day, greater than or equal to 3 days, greater than or equal to 5 days, greater than or equal to 7 days, greater than or equal to 10 days, greater than or equal to 2 weeks, greater than or equal to 4 weeks, greater than or equal to 6 weeks, greater than or equal to 12 weeks, greater than or equal to 26 weeks, or greater than or equal to 52 weeks. In some embodiments, the (nano)diamond particles are configured for residence in an organ of a subject for less than or equal to 100 weeks, less than or equal to 52 weeks, less than or equal to 26 weeks, less than or equal to 12 weeks, less than or equal to 6 weeks, less than or equal to 4 weeks, less than or equal to 2 weeks, less than or equal to 10 days, less than or equal to 7 days, less than or equal to 5 days, or less than or equal to 3 days. Combinations of the above-referenced ranges are also possible (e.g., greater than 1 day and less than 100 weeks, greater than 5 days and less than 26 weeks, greater than 6 weeks and less than 52 weeks). Other ranges are also possible. In some embodiments, the (nano)diamond particles may be configured to reside in the organ of the subject for the lifespan of the subject. Advantageously, the (nano)diamond particles described herein may reside in an organ of a subject without toxic or detrimental physiological effects.

In certain embodiments, (nano)diamond particles may be captured by an organ internal to a subject. In some embodiments, the (nano)diamond particles may further (e.g., spontaneously) organize or aggregate within a subject or within an organ internal to a subject. In some embodiments, (nano)diamond particles may form aggregates e.g., within an organ such as the liver. In some embodiments, diamond nanoparticles (e.g., (nano)diamond particles) may form aggregates within, e.g., the pancreas and/or pancreatic cells. An example of aggregation is shown in Example 3, below. In some cases, these aggregates advantageously may help to monitor the progression of a condition or disease within a subject and/or provide long term delivery of a therapeutic agent.

As described herein, (nano)diamond particles may be administered to a subject. In some cases, the plurality of (nano)diamond particles are administered surgically (e.g., implanted) and/or injected (e.g., into the systemic circulation, intraocular, into the spinal system cord or fluids, e.g., via syringe). In certain embodiments, the plurality of (nano)diamond particles may be administered orally, rectally, vaginally, nasally, ureteral or by inhalation to the subject (e.g., within a capsule).

In some embodiments, administration of the (nano)diamond particles is via injection such as intravenous injection. For example, an injection component associated with a reservoir comprising the (nano)diamond particles may be used. In some embodiments, the injection component is a needle and the associated reservoir is a syringe. The needle may be of any size or gauge appropriate for administering a composition to a subject. The syringe may be of any size or volume appropriate for containing a particular amount of composition to be administered to a subject. In some embodiments, the injection component is a pipette. Those skilled in the art will be aware of other injection components suitable for administering a composition as described herein to a subject, as the disclosure is not so limited.

In some embodiments, the reservoir comprises an intravenous carrier fluid and a plurality of (nano)diamond particles suspended within the intravenous carrier fluid. Non-limiting examples of suitable intravenous carrier fluids include saline (e.g., 9% normal saline, 45% normal saline), lactated Ringers, and aqueous dextrose (e.g., 5% dextrose in water).

In some embodiments, (nano)diamond particles (e.g., the plurality of (nano)diamond particles comprising a therapeutic agent bound to the (nano)diamond particles) may be administered to a subject (e.g., for the detection of an analyte (e.g., a biological element of physiological of pathological identity) suspected of being present in the subject). For example, in some cases, the plurality of (nano)diamond particles comprising the therapeutic agent may be administered to the subject and, upon detection of an emission (e.g., fluorescent emission, near infrared emission, etc.) of the (nano)diamond particles, confirm the presence of the therapeutic agent in the subject.

In some embodiments, a species (e.g. a therapeutic agent) is bound to a (nano)diamond particles or a plurality of (nano)diamond particles. In some embodiments, (nano)diamond particles are associated with (e.g., bound to) the species via functionalization of the (nano)diamond particle. For example, in some embodiments, a (nano)diamond particle is associated with a species via formation of a bond, such as an ionic bond, a covalent bond, a hydrogen bond, Van der Waals interactions, and the like. The covalent bond may be, for example, a carbon-carbon, carbon-oxygen, oxygen-silicon, sulfur-sulfur, phosphorus-nitrogen, carbon-nitrogen, metal-oxygen, or other covalent bond. The hydrogen bond may be, for example, between hydroxyl, amine, carboxyl, thiol, and/or similar functional groups. For example, the species may further include a functional group, such as a thiol, aldehyde, ester, carboxylic acid, hydroxyl, and the like, wherein the functional group forms a bond with the (nano)diamond particle. In some embodiments, a function group is bound to the (nano)diamond particles (e.g., capable of binding to the therapeutic agent). In some cases, the species may be an electron-rich or electron-poor moiety wherein interaction between the (nano)diamond particle and the species comprises an electrostatic interaction.

In some embodiments, a species (e.g. a therapeutic agent) is associated with a functionalized (nano)diamond particle comprising a —COOH, —OH, —NH₂, —SH, or —C═O functional group by reacting the functionalized (nano)diamond particle and the species in the presence of a cross-linking agent. Non-limiting examples of suitable cross-linking agents include carbodiimides such as 1-ethyl-3-[3-dimethylaminopropyl]carbodiimide hydrochloride (EDC); amine-reactive compounds such as N-Hydroxysuccinimide ester, imidoester, and hydromethylphosphine; sulfhydryl-reactive compounds such as maleimide, pyridyl disulfides, and iodoacetyl; aldehyde-reactive compounds such as hydrazide and alkoxyamine; and photoreactive cross-linking agents such as aryl azides and diazirine. Other cross-linking agents are also possible. Those of ordinary skill in the art would be capable of selecting suitable cross-linking agents based upon the type of species selected and the teachings of this specification.

Examples of suitable (nano)diamond particles are described in more detail in co-owned International Patent Application No. PCT/US2017/050257, filed Sep. 6, 2017, entitled “(NANO)DIAMOND PARTICLES AND RELATED DEVICES AND METHODS” which is incorporated herein by reference in its entirety for all purposes.

As described above and herein, in some embodiments, (nano)diamond particles may be used for imaging. For example, in some embodiments, the (nano)diamond particles may emit (i.e. fluorescence) a characteristic emission which may be detected by a detector. In some embodiments, a detector may be positioned proximate a region of a subject suspected of containing the (nano)diamond particles. For example, the plurality of (fluorescent) (nano)diamond particles functionalized with a species may be administered to a subject, and the detector may be positioned proximate the subject such that any (nano)diamond particles may be detected (e.g., via an emission of the (nano)diamond particles).

Any suitable detector may be used with the devices and methods described herein. For example, in some embodiments, the detector may be an optical detector (e.g., fluorescence detectors, visible light and/or UV detectors, near infrared detectors, microscopes, MRI, CT scanners, x-ray detectors).

As described herein, a (nano)diamond particle is an aggregate of carbon atoms where at the core lies a diamond cage composed mainly of carbon atoms. Although (nano)diamond particles comprise diamond, other phases or allotropes of carbon may be present, such as graphite, graphene, fullerene, etc. A single (nano)diamond particle may comprise a single form of carbon in some embodiments. In other embodiments, more than one form of carbon may comprise a (nano)diamond particle.

In some embodiments, a plurality of diamond particles may have an average largest cross-sectional dimension (e.g. a diameter) of 2 μm or less. While much of the description is generally related to nanodiamond particles (i.e. diamond particles having a largest cross-sectional dimension of less than 1000 nm), those of ordinary skill in the art would understand, based upon the teachings of this specification, that diamond particles having larger cross-sectional dimensions (e.g., greater than or equal to 1000 nm) are also possible. For example, in some embodiments, the plurality of diamond particles may have an average largest cross-sectional dimension of less than 2 μm (e.g., less than or equal to 1800 nm, less than or equal to 1600 nm, less than or equal to 1400 nm, less than or equal to 1200 nm, less than or equal to 1000 nm, less than or equal to 900 nm less than or equal to 800 nm, less than or equal to 700 nm, less than or equal to 600 nm, less than or equal to 400 nm, less than or equal to 200 nm, less than or equal to 180 nm, less than or equal to 160 nm, less than or equal to 140 nm, less than or equal to 120 nm, less than or equal to 100 nm, less than or equal to 80 nm, less than or equal to 60 nm, less than or equal to 40 nm, or less than or equal to 20 nm). In some cases, the plurality of diamond particle may have an average largest cross-sectional dimension of greater than or equal to 10 nm, greater than or equal to 20 nm, greater than or equal to 40 nm, greater than or equal to 60 nm, greater than or equal to 80 nm, greater than or equal to 100 nm, greater than or equal to 120 nm, greater than or equal to 140 nm, greater than or equal to 160 nm, greater than or equal to 180 nm, greater than or equal to 200 nm, greater than or equal to 400 nm, greater than or equal to 600 nm, greater than or equal to 700 nm, greater than or equal to 800 nm, greater than or equal to 900 nm, greater than or equal to 1000 nm, greater than or equal to 1200 nm, greater than or equal to 1400 nm, greater than or equal to 1600 nm, or greater than or equal to 1800 nm. Combinations of the above-referenced ranges are also possible (e.g., less than 2 μm and greater than or equal to 10 nm, less than or equal to 1400 nm and greater than or equal to 1000 nm). Other ranges are also possible. Those of ordinary skill in the art are capable of selecting suitable methods for determining the average cross-sectional dimension of a plurality of diamond based upon the teachings of this specification. In an exemplary set of embodiments, the plurality of diamond particles have an average largest cross-sectional dimension of less than or equal to 900 nm and greater than or equal to 700 nm. In some embodiments, diamond particles may form aggregate structures with other diamond particles (e.g., at a location internal to the subject). An aggregate of diamond particles, in some embodiments, may have a largest cross-sectional dimension greater than or equal to 1 μm (e.g. greater than or equal to 1 μm, greater than or equal to 5 μm, greater than or equal to 10 μm, greater than or equal to 20 μm, greater than or equal to 30 μm, greater than or equal to 40 μm, greater than or equal to 50 μm, greater than or equal to 60 μm, greater than or equal to 70 μm, greater than or equal to 80 μm, greater than or equal to 90 μm) and less than or equal to 100 μm (e.g. less than or equal to 100 μm, less than or equal to 90 μm, less than or equal to 80 μm, less than or equal to 70 μm, less than or equal to 60 μm, less than or equal to 50 μm, less than or equal to 40 μm, less than or equal to 30 μm, less than or equal to 20 μm, less than or equal to 10 μm, less than or equal to 5 μm, less than or equal to 1 μm). Combinations of the above-referenced ranges are also possible (e.g., greater than or equal to 1 micron and less than or equal to 50 microns, greater than or equal to 1 micron and less than or equal to 100 microns). Other ranges are also possible.

In some embodiments, (nano)diamond particles may form relatively large aggregate structures with other (nano)diamond particles (e.g., at a location internal to the subject). For example, in some embodiments, the aggregate of (nano)diamond particles has a largest cross-sectional dimension of greater than or equal to 100 microns, greater than or equal to 200 microns, greater than or equal to 500 microns, greater than or equal to 1000 microns, greater than or equal to 2000 microns, greater than or equal to 5000 microns, or greater than or equal to 7500 microns. In some embodiments, the aggregate of (nano)diamond particles has a largest cross-sectional dimension of less than or equal to 10000 microns, less than or equal to 7500 microns, less than or equal to 5000 microns, less than or equal to 2000 microns, less than or equal to 1000 microns, less than or equal to 500 microns, or less than or equal to 200 microns. Combinations of the above-referenced ranges are also possible (e.g., greater than or equal to 1 micron and less than or equal to 10000 microns, greater than or equal to 100 microns and less than or equal to 10000 microns, greater than or equal to 500 microns and less than or equal to 5000 microns, greater than or equal to 1000 microns and less than or equal to 10000 microns). Other ranges are also possible.

In some embodiments, the (nano)diamond particles may emit electromagnetic radiation. In some embodiments, the emission is a fluorescent emission. In certain embodiments, the wavelength of the emission is greater than or equal to 250 nm, greater than or equal to 300 nm, greater than or equal to 350 nm, greater than or equal to 400 nm, greater than or equal to 450 nm, greater than or equal to 500 nm, greater than or equal to 550 nm, greater than or equal to 600 nm, or greater than or equal to 650 nm. In certain embodiments, the wavelength of the emission is less than or equal to 700 nm, less than or equal to 650 nm, less than or equal to 600 nm, less than or equal to 550 nm, less than or equal to 500 nm, less than or equal to 450 nm, less than or equal to 400 nm, less than or equal to 350 nm, or less than or equal to 300 nm. Combinations of the above-referenced ranges are also possible (e.g., greater than or equal to 250 nm and less than or equal to 700 nm). Other ranges are also possible.

In certain embodiments, the emission is a near infrared emission. In some embodiments, the wavelength of the emission is greater than 700 nm, greater than or equal to 750 nm, greater than or equal to 800 nm, greater than or equal to 850 nm, greater than or equal to 900 nm, or greater than or equal to 950 nm. In certain embodiments, the wavelength of the emission is less than or equal to 1000 nm, less than or equal to 950 nm, less than or equal to 900 nm, less than or equal to 850 nm, less than or equal to 800 nm, or less than or equal to 750 nm. Combinations of the above-Substitute referenced ranges are also possible (e.g., greater than 700 nm and less than or equal to 1000 nm). Other ranges are also possible.

In an exemplary embodiment, the (nano)diamond particles have a near infrared emission (e.g., greater than or equal to 650 nm and less than or equal to 750 nm) and an average largest cross-sectional dimension of about 700-900 nm. Other combinations of emissions and cross-sectional dimensions are also possible.

In some embodiments, the (nano)diamond particle may emit a fluorescent and/or near infrared emission upon excitation by electromagnetic radiation having a particular wavelength. For example, in some embodiments, the (nano)diamond particle may be exposed to electromagnetic radiation having a wavelength of greater than or equal to 250 nm, greater than or equal to 300 nm, greater than or equal to 350 nm, greater than or equal to 400 nm, greater than or equal to 450 nm, greater than or equal to 500 nm, greater than or equal to 550 nm, greater than or equal to 600 nm, greater than or equal to 650 nm, greater than or equal to 700 nm, greater than or equal to 750 nm, greater than or equal to 800 nm, greater than or equal to 850 nm, greater than or equal to 900 nm, or greater than or equal to 950 nm (e.g., such that the (nano)diamond particle emits a fluorescent emission and/or near infrared emission in one of the above-referenced ranges). In certain embodiments, the (nano)diamond particle may be exposed to electromagnetic radiation having a wavelength of less than or equal to 1000 nm, less than or equal to 950 nm, less than or equal to 900 nm, less than or equal to 850 nm, less than or equal to 800 nm, or less than or equal to 750 nm, less than or equal to 700 nm, less than or equal to 650 nm, less than or equal to 600 nm, less than or equal to 550 nm, less than or equal to 500 nm, less than or equal to 450 nm, less than or equal to 400 nm, less than or equal to 350 nm, or less than or equal to 300 nm. Combinations of the above-referenced ranges are also possible (e.g., greater than or equal to 250 nm and less than or equal to 1000 nm, greater than or equal to 550 nm and less than or equal to 650 nm). Other ranges are also possible.

Without wishing to be bound by theory, in some cases, the (nano)diamond particles described herein may be auto-fluorescent (e.g., the (nano)diamond particles emit fluorescent light e.g., after absorption of electromagnetic radiation). In some cases, the (nano)diamond particles may comprise one or more atomistic-type defects (e.g., a point defect such as a nitrogen-vacancy (NV) center, a point defect such as a nitrogen-vacancy-nitrogen (NVN) defect, combinations thereof) which result in near-infrared fluorescence and/or photoluminescence that may be detected and/or quantified. Other defects are also possible (e.g., Gadolinium, Europium, iron, Si-vacancy defects). In certain embodiments, the (nano)diamond particles fluoresce in response to an applied electromagnetic radiation.

For example, in some embodiments, the (nano)diamond particle may be excited (e.g., by applying electromagnetic radiation having a first wavelength) such that the (nano)diamond particle emits a detectable emission (e.g., an electromagnetic radiation having a second wavelength, different than the first wavelength). In a particular set of embodiments, if an analyte is present in a sample, the analyte binds to the (nano)diamond particle (e.g., binds to a species bound to the (nano)diamond particle) such that an emission from the (nano)diamond particle may be detected and/or quantified. In some cases, detection of an emission of (nano)diamond particles in a subject may indicate that the (nano)diamond particles are bound to the suspected analyte. In some such cases, the emission may be quantified (e.g., to determine the relative amount of analyte present in the subject).

As described herein, certain embodiments comprise a therapeutic agent bound to (nano)diamond particles. According to some embodiments, the therapeutic agent may be one or a combination of therapeutic, diagnostic, and/or enhancement agents, such as drugs, nutrients, microorganisms, in vivo sensors, and tracers. In some embodiments, the therapeutic agent is a nutraceutical, prophylactic or diagnostic agent. While much of the specification describes the use of therapeutic agents, other agents listed herein are also possible.

Agents can include, but are not limited to, any synthetic or naturally occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals, Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In certain embodiments, the therapeutic agent is one or more specific therapeutic agents. As used herein, the term “therapeutic agent” or also referred to as a “drug” refers to an agent that is administered to a subject to treat a disease, disorder, pathology, or other clinically recognized condition, or for prophylactic purposes, and has a clinically significant effect on the body of the subject to ameliorate, treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C.A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, anti-epileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, anti-parasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

In some embodiments, the therapeutic agent is one or more anticancer drugs (e.g., chemotherapy drugs).

Non-limiting examples of suitable anti-cancer therapeutic agents include alkylating agents (e.g., Cyclophosphane, Busulfan, cisplatin), antimetabolic compounds (e.g., folic acid analogs-methotrexate), purine analogs (e.g., mercaptopurine, Pentostatin), pyrimidine analogs (e.g., 5-fluor uracil), vinca alkaloids, camptothecins, proteaome inhibitors (e.g., Gefitinib), anthracyclines (e.g., doxorubicin), hormones (e.g., steroids), biological adjuvants treatments (e.g., antibodies, Herceptin), adjuvant treatments (e.g., BRAF, Melanoma), dabrafenib/Tafinlar; Trametinib/Mekinist), biospecific antibodies, blinatumomab/Blincyto, chemolabeled antibodies, and Brentuximab.

Further non-limiting examples of suitable anti-cancer therapeutic agents include cyclophosphamide, methotrexate, 5-fluorouracil, vinorelbine, doxorubicin, docetaxel, bleomycin, vinblastine, dacarbazine, mustine, vincristine, procarbazine, prednisolone, etoposide, cisplatin, epirubicin, capecitabine, methotrexate, vincristine, folinic acid, oxaliplatin, gemcitabine, ifosfamide, and etoposide. In an exemplary set of embodiments, the anti-cancer therapeutic agent is doxorubicin. In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell receptors or Il-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In certain embodiments, the therapeutic agent is a hormone or derivative thereof. Non-limiting examples of hormones include insulin, growth hormone (e.g., human growth hormone), vasopressin, melatonin, thyroxine, thyrotropin-releasing hormone, glycoprotein hormones (e.g., luteinzing hormone, follicle-stimulating hormone, thyroid-stimulating hormone, TSH), eicosanoids, estrogen, progestin, testosterone, estradiol, cortisol, adrenaline, and other steroids.

In some embodiments, the therapeutic agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In some cases, the therapeutic agent is a small molecule drug having molecular weight between 200 Daltons and 400 Daltons, between 400 Daltons and 1000 Daltons, or between 500 Daltons and 2500 Daltons.

In some embodiments, the therapeutic agent is selected from the group consisting of active pharmaceutical agents such as nucleic acids, peptides, bacteriophage, DNA, mRNA, aptamers, human growth hormone, monoclonal antibodies, adalimumab, epinephrine, GLP-1 Receptor agonists, semaglutide, liraglutide, dulaglitide, exenatide, factor VIII, small molecule drugs, progestin, vaccines, subunit vaccines, recombinant vaccines, polysaccharide vaccines, and conjugate vaccines, toxoid vaccines, influenza vaccine, shingles vaccine, prevnar pneumonia vaccine, mmr vaccine, tetanus vaccine, hepatitis vaccine, HIV vaccine Ad4-env Clade C, HIV vaccine Ad4-mGag, DNA vaccines, RNA vaccines, etanercept, infliximab, filgastrim, glatiramer acetate, rituximab, bevacizumab, any molecule encapsulated in a nanoparticle, epinephrine, lysozyme, glucose-6-phosphate dehydrogenase, other enzymes, certolizumab pegol, ustekinumab, ixekizumab, golimumab, brodalumab, guselluab, secikinumab, omalizumab, tnf-alpha inhibitors, interleukin inhibitors, vedolizumab, octreotide, teriperatide, CRISPR cas9, oligonucleotides, and ondansetron.

The therapeutic agent may be present in a composition comprising the (nano)diamond particles in any suitable amount (e.g., a therapeutically effective amount). For example, in some embodiments, the therapeutic agent (e.g., the anti-cancer therapeutic agent) is present in the composition in an amount of greater than or equal to 1 microgram, greater than or equal to 2 micrograms, greater than or equal to 5 micrograms, greater than or equal to 10 micrograms, greater than or equal to 11 micrograms, greater than or equal to 12 micrograms, greater than or equal to 15 micrograms, greater than or equal to 20 micrograms, greater than or equal to 25 micrograms, greater than or equal to 30 micrograms, greater than or equal to 40 micrograms, greater than or equal to 50 micrograms, greater than or equal to 60 micrograms, greater than or equal to 70 micrograms, great than or equal to 75 micrograms, greater than or equal to 80 micrograms, greater than or equal to 90 micrograms, greater than or equal to 100 micrograms, greater than or equal to 125 micrograms, greater than or equal to 150 micrograms, or greater than or equal to 200 micrograms per 1 milligram of (nano)diamond particles. In some embodiments, the therapeutic agent (e.g., the anti-cancer agent) is present in the composition in an amount less than or equal to 250 micrograms, less than or equal to 200 micrograms, less than or equal to 150 micrograms, less than or equal to 100 micrograms, less than or equal to 90 micrograms, less than or equal to 80 micrograms, less than or equal to 75 micrograms, less than or equal to 70 micrograms, less than or equal to 60 micrograms, less than or equal to 50 micrograms, less than or equal to 40 micrograms, less than or equal to 30 micrograms, less than or equal to 25 micrograms, less than or equal to 20 micrograms, less than or equal to 15 micrograms, less than or equal to 12 micrograms, less than or equal to 11 micrograms, less than or equal to 10 micrograms, less than or equal to 5 micrograms, or less than or equal to 2 micrograms per 1 milligram of (nano)diamond particles. Combinations of the above referenced ranges are also possible (e.g., greater than or equal to 1 microgram and less than or equal to 250 micrograms, greater than or equal to 10 micrograms and less than or equal to 100 micrograms, greater than or equal to 11 micrograms and less than or equal to 75 micrograms). Other ranges are also possible.

While much of the description herein is in the context of (fluorescent) (nano)diamond particles, those of ordinary skill in the art would understand, based upon the teachings of this specification, that other particles are also possible. For example, in some embodiments, the device may comprise a particle such as a nanoparticle (e.g., a silica nanoparticle, a sapphire nanoparticle, a garnet nanoparticle, a ruby nanoparticle, a quantum dot, a quantum dot-polymer composite) having an emission in one of the above referenced ranges associated with a species (e.g., a species capable of binding to one or more target analytes). In some cases, the particle may be auto-fluorescent. In other cases, the particle may be functionalized with (e.g., associated with) a fluorescent molecule.

In an illustrative embodiment, fluorescent (nano)diamond particles administered to a subject gain access to the liver cells (e.g., hepatocytes, kupfer cells), as well as other cells (e.g., endothelium) where the deposition of (nano)diamond particles in the liver is substantially immediate (upon (nano)diamond particles injections). In some such embodiments, the presence of the (nano)diamond particles in the liver is prolonged e.g., a single injections could provide a sustained presence of particles at least over 12 weeks. In some embodiments, (nano)diamond particles present in the liver do not convey adverse effects on the normal liver cells (e.g., measured at least after 3 months). (nano)diamond particles and/or an associated species (e.g., a chemical and/or organic additive functionalized on the (nano)diamond particle) may, in some cases, find facilitated entrance and increased accumulation within cancer cells (over the normal liver cells). In some embodiments, therapeutic agents having anti-cancer properties, when tagged onto the fluorescent (nano)diamond particles, may arrest cancer cells growth (e.g., diminishing the metastatic scale and its progression). In some such embodiments, the (nano)diamond particles (e.g., bound to therapeutic agents) may advantageously afford longer “progression free disease” periods and reduced mortality. In some embodiments, without wishing to be bound by theory, diminishing the metastatic burden the liver, may advantageously contribute to betterment of liver function (a severe cause of morbidity on its own).

### EXAMPLES

The following examples are intended to illustrate certain embodiments of the present invention, but do not exemplify the full scope of the invention.

### Example 1

Rodent POC (efficacy) studies raised an important caveat, i.e. anatomical considerations suggest that recording NIR from locales within the body pose challenges associated with opacity and auto-fluorescence in an organ-and tissue-dependent manner. To optimize NIR signal captured from within the body in a location such as the carotid artery (in humans, 15-20 mm from skin surface), large nanodiamond particles were selected: FNDP-(NV)-Z-average˜800 nm (fluorescent nanodiamond particles having a nitrogen-vacancy and an average largest cross-sectional dimension of about 800 nm), a strain of particles that possess superior NIR emission (650˜720 nm) over smaller particles of the same strain as well as over the NVN “color center” strain. The particles ‘homed’ to the target (intra-vascular blood clots) shortly after infusion (envisioning the targeted clinical procedure) and considering the rapid clearance of FNDP-(NV) from the circulation (50% clearance from serum within 4 minutes), likely via rapid uptake by the reticuloendothelial system of the liver, the loading dose of the FDNP-(NV) was explored to afford target imaging.

Administration of particles of the size chosen (Z-average˜700/800 nm) directly into the systemic circulation may result in prolonged, if not indefinite, particles residency within organs due to unlikely excretion routes (urinary system or the hepatobiliary system). Such concerns are supported by in vitro studies where extended residency of other similarly sized particles in cells (in culture) suggested interference with biological functions and viability.

The examples herein, designed to explore FDNP-(NV) distribution in rat organs upon both short and long-term exposure demonstrated principle deposition of particles in the liver with secondary deposition to the spleen while other organs shared none or only a minor fraction (e.g. less than 5%). Interestingly, the large deposit of FDNP-(NV) in the liver 5 days after exposure remained unchanged in the 14-days and 12-weeks post-exposure studies.

The present investigation on the intra-hepatic topological distribution of FNDP-(NV) was generally carried out by conventional fluorescent microscopy (FM) and confocal fluorescent microscopy (CFM) of liver slices (5-50 μm). Furthermore, an in vitro investigation on the kinetics of FNDP-(NV) uptake into cells, such as human hepatic carcinoma cells (HepG2) and human umbilical vein endothelial cells (HUVEC) commonly used as proxies for hepatocytes and vascular endothelium, respectively, was performed. The in-vitro results demonstrated the capacity of liver cells to incorporate FDNP-(NV) as well the subcellular distribution of engulfed particles.

### Material and Methods

FNDP-(NV)-Z-average˜700/800 nm: source and functionalization

FNDP-(NV)-Z-average˜700/800 nm functionalized with carboxyl moieties were purchased from ADAMAS Nanotechnologies (Raleigh, NC, USA). The physical properties of the FNDP-(NV) were determined by dynamic light scattering on a Zetasizer Nano (Malvern) as having an average diameter of 858±47 nm and Z-potential of −56 mV, as reported previously. Sterile and BSA blocked FNDP-(NV) were used in the cell based studies.

Liver specimens were obtained as follows: Briefly, Sprague-Dawley rats were injected into the femoral vein at 60 mg/Kg of FNDP-(NV) suspension in 2 mL PBS over 2-3 minutes. After 12 weeks, the animals were sacrificed by exsanguination while under deep (5% isoflurane) anesthesia, perfused with 10 mL sterile saline to minimize residual blood in the organs vasculature and further by perfusion of 4% paraformaldehyde in saline for organ preservation. Organs were carefully dissected, suspended in excess of 10% neutral buffered formaldehyde (10% NBF). Liver specimens were then processed and embedded in paraffin for sectioning into 5 or 50 μm slices for analysis by, fluorescence microscopy (FM) or confocal fluorescent microscopy (SCM), respectively. The liver specimens evaluated in this study were discrete and holistic lobes dissected after whole organ imaging by IVIS. For histopathology examination 5 μm sections of liver specimens were stained with Hematoxylin and Eosin (H&E) and Masson's trichrome by independent histopathology evaluation.

Rat liver specimens were embedded in paraffin and sectioned at 5 or 50 μm thickness as described previously. In brief, slides were de-paraffinized by three consecutive rinses (5 min each) with xylene followed by two consecutive rinses (10 min each) of 100%, 95%, 70% and 50% ethanol and two final washes with deionized water. Cellular actin filaments were stained with FITC-phalloidin. Briefly, slides were permeabilized by incubation with 0.4% Triton X-100 in PBS on ice for 10 min. The slides were then washed 3 times with PBS at room temperature and immersed in FITC-phalloidin (6 μM in PBS) for 1 hour. The slides were washed three times with PBS and mounted with mounting buffer containing DAPI to stain nuclei. The 5 μm thick slices were analyzed in a fluorescence microscope using 10× and 40× (oil immersion) objectives. The green fluorescence filter set was used to detect the FITC-phalloidin stained microfilaments, the red fluorescent filter to was used detect FNDP-(NV) and the blue, fluorescent filters to detect DAPI stained cell nuclei.

Total panoramic views of sagittal sections of the liver were constructed by ‘stitching’ 4× images using an FSX100 microscope. 50 μm sections were stained with FITC-phalloidin for visualization of actin filaments imaged in the green channel, and sections were imaged in the red channel for visualization of FNDP-(NV). Images were collected digitally and further processed with ImageJ 1.51e (NIH, Bethesda MD, USA). In order to improve visualization of FNDP-(NV), which were only a few pixels in size at the ultra-low magnification, particles were magnified by thresholding the red channel using the Maximum Entropy method and dilating the result three times.

FNDP-(NV) presence in cells after image thresholding, but not dilating, was also quantified using the Analyze Particles function in ImageJ. Groups of FNDP-(NV), detected as a single continuous mass (agglomerate) at 4×, were counted and sized. The size distribution by number histogram was constructed to demonstrate the distribution of FNDP-(NV) agglomeration sizes detected within the micrographs, where line height corresponds to the portion of particles detected by diameter. As large numbers of small agglomerations can account for a small number of total particle mass, size distribution by number can be considered biased to magnify the prevalence of small particle sizes. To reduce this bias, a second histogram of the size distribution by cross-section area was also constructed where line height correlates with portion of total NIR fluorescing area.

Confocal images of liver slices (10-50 μm) were taken using an FV1000 scanning confocal microscope and imaged in Fluoview software (v4.2.2.9 Olympus) using a 60× oil immersion objective. For 3D reconstruction, confocal stacks were taken with an image every 0.5 μm through the thickness of the tissue. Nuclei were visualized by DAPI staining with a 405 nm excitation and 425-460 nm emission; the actin cytoskeleton was visualized by FITC-phalloidin with a 488 nm excitation and 400-500 nm emission. The NIR fluorescence emitted from FNDP-(NV) was visualized with an excitation of 543 nm and an emission of 655-755 nm. 2D maximum intensity projection and cross-sectional views were prepared in Fluoview. Three dimensional views were reconstructed in ImageJ via the Volume Viewer plugin.

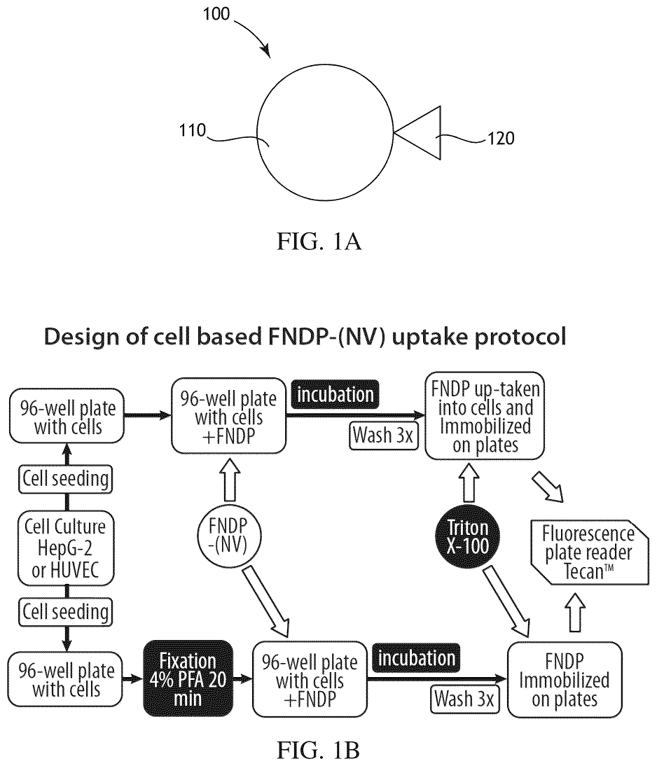

The HepG-2 (human liver hepatocellular carcinoma) cell line was purchased from American Type Culture Collection (ATTC) (Manassas, VA, USA) and cultured in Eagle's Minimum Essential Medium (EMEM, ThermoFisher Scientific) containing 10% fetal bovine serum (FBS). Primary human umbilical vein endothelial cells (HUVEC) were purchased from Lonza (Basel, Switzerland) and cultured in EGM-2 MV media. HUVEC were used for experiments in passages 5-8. Uptake of FNDP-(NV) by either cell line was performed according to previously published protocols with some modifications, as illustrated in FIG. 1. Briefly, cells were seeded into 2 96-well plates (2×10⁴ cells per well) and allowed to grow to 90% confluence. Media was removed from one plate (background control) and 100 μL of 4% paraformaldehyde (PFA) in PBS was added to fix the cells. The control plate was then incubated for 20 min at room temperature and washed 3 times with cell culture media. Subsequently, media was removed from each well in both plates (fixed control and live sample), replaced with 100 μL of media containing FNDP-(NV) at 0.025, 0.05, and 0.1 mg/mL as indicated, and allowed to incubate for 0.5-20 hr. Both plates were washed 3 times with Hanks'balanced salt solution (HBSS, ThermoFisher, and Waltham, MA, USA) containing calcium and magnesium to remove excess particles. Cells were then lysed by addition of 100 μL of 0.5% Triton X-100 and overnight incubation at room temperature on orbital shaker. Plates were read using spectrophotometer (Infinite M200 Pro, Tecan AG, Männedorf, Switzerland) for FNDP-(NV) associated NIR signal (excitation 570 nm, emission 670 nm). Fluorescence obtained from FNDP-(NV) attached to the control plate with PFA fixed cells was deducted from fluorescence measured from live (active) cells.

Cells were grown in 8-well chambers slides (ThermoFisher) up to 70% confluence. Cell were treated with FNDP-(NV) at 0.05 mg/mL, and incubated for 2 or 20 hrs., and fixed in 4% PFA as described above. Following cell fixation and permeabilization, cells were stained with FITC-Phalloidin as described above. Chambers were removed from the slide, and mounting was completed using buffer containing DAPI (Vectashield) and cover glass affixed by nail polish. Slides were then analyzed on the FM Olympus IX81 at 10× or 40×, using the green, red, and blue filter cubes as described in Fluorescent microscopy of preserved liver slices.

Data are presented as mean±SD. Statistical analyses were done by ANOVA (where appropriate) and Student's t-test using SigmaPlot software (SigmaPlot® 12 SPSS; Systat Software Inc., San Jose CA, USA). Statistical significance was established at P<0.05 for the number of independent studies performed.

### Results

### Fluorescence Microscopic (FM) and Panoramic Analysis of Preserved Liver Slices

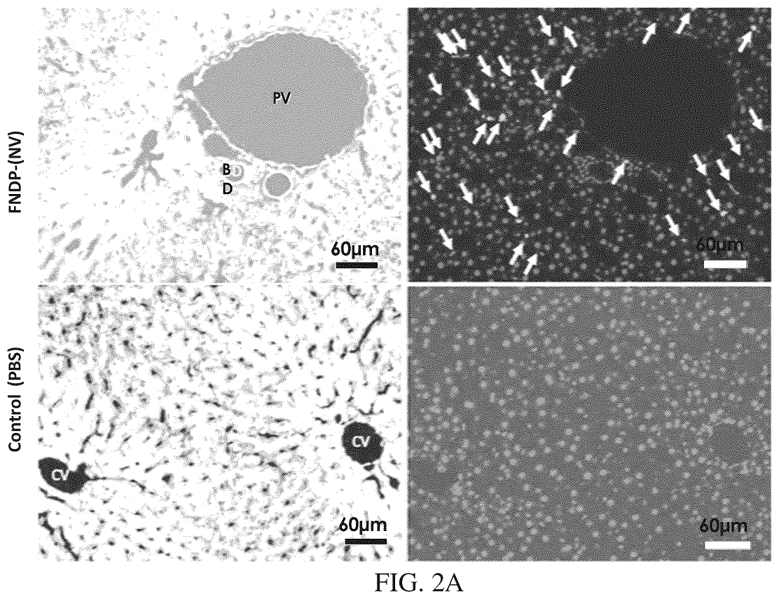

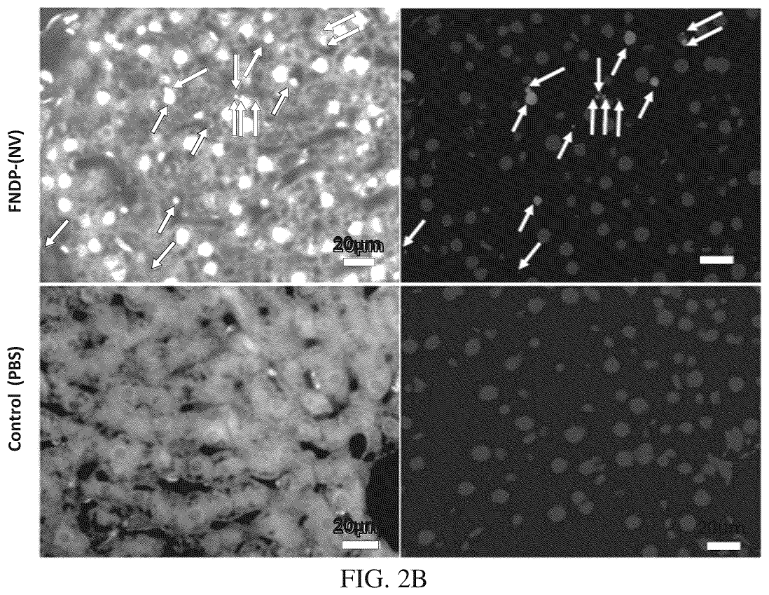

FIG. 2A illustrates the distribution of FNDP-(NV) within a 5 μm slice of liver tissue imaged at 160× and 400× magnifications. Two representative regions have been selected; one (FIG. 2A) where vascular elements are present, second (FIG. 2B) an area of parenchyma cells only. The upper panels represent tissue obtained from animals 12 weeks after intravenous (IV) administration of FNDP-(NV) and the lower panel from a vehicle (PBS) control animal. FNDP-(NV) (imaged in red, shown in a shade of grey) can be visualized over the DAPI counter stain in the right panel as identified by white arrows. Large agglomerations of 5-10 μm are clearly noted, as well as particles of very small size. To assess distribution within or between cells, the sections were stained with FITC-phalloidin as shown in the left panels. The corresponding yellow (red-over-green) can be visualized for larger aggregates, indicating possibility of particle endocytosis (left panels (FIGS. 2A and 2B). In FIG. 2A, red fluorescence of very small aggregates can be spotted in proximity of nuclei that possibly represent portal vein (PV) endothelium but most are distributed in the parenchyma where it is rather difficult to discern venous space from parenchyma cells location.

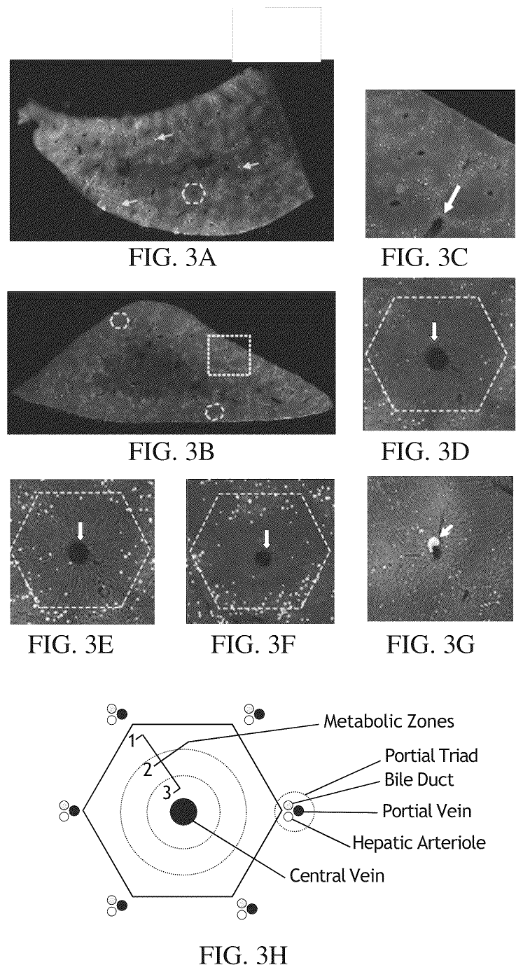

FIGS. 3A-3H presents an analysis at multiple magnifications of a complete sagittal section from 2 different FNDP-(NV) treated rats. Due to the very low effective magnification of the ‘stitched’ image, the red channel, sensitive to the NIR fluorescence of FNDP-(NV), has been magnified by binary thresholding and dilating as indicated in methods. This allows qualitative visualization of even single pixel FNDP-(NV). FIGS. 3A and 3B depict particles scattered across the complete “panoramic landscape” yet with apparent differential densities in their distribution within the core hepatic lobule unit. For ease of visualization, a select number of hepatic lobules are indicated by “hexagons”. Particles can also be easily spotted in the venous system (yellow boxes). A magnified view, suitable for visualization without enhancement, of a set of four hepatic lobules (region indicated by blue dotted rectangle in FIG. 3B) is presented in FIG. 3C, which illustrates apparent heterogeneity of particle distribution within the hepatic lobule. A higher magnification of a single lobule (yellow hexagon from FIG. 3A) is presented in FIG. 3D. To enhance visualization, a higher magnification of one representative lobule from each animal (as indicated by yellow hexagon in FIG. 3A and B) is presented in FIG. 3E and F. After thresholding and dilating, better illustration of the uneven distribution of particles across the “hexagon” formation of the hepatic lobule is easily noticed Particle presence appears enriched at the “hexagonal” periphery (for landmarks, red arrows mark central veins), though some particles are clearly present even beside the central vein. FIG. 3G and H depict venous systems (yellow squares in FIG. 3A) with large aggregation of particles (white arrow) that are attached to the wall but protrudes significantly into the vessel lumen (visualized by the yellow-red transition) accounting for 35% and 48% of the vessel cross sectional areas in the 2 examples, respectively. FIG. 3I provides a scheme of the general orientation of the structure of the hepatic lobule including the primary metabolic zones.

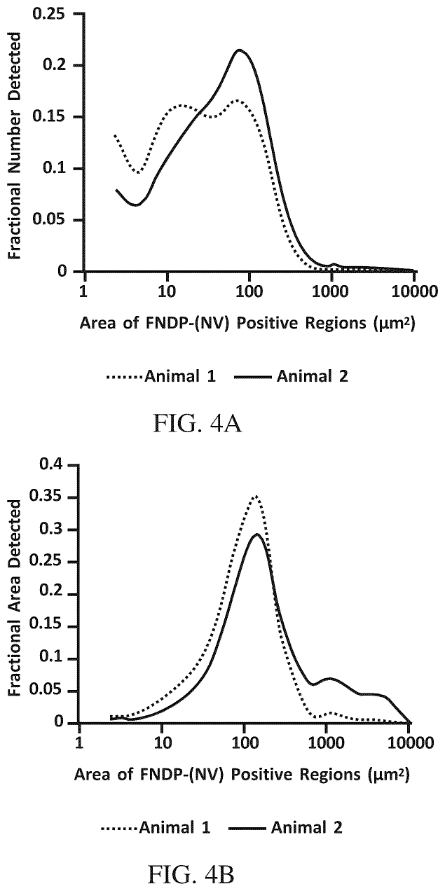

The size of FNDP-NV positive regions in the liver “panoramic” view is highly variable as indicated above. To quantify this distribution, a histogram of FNDP-NV positive regions is presented in FIG. 4. The distribution of the regions by number in FIG. 4A demonstrates large numbers of FNDP-NV positive areas from a single pixel, up to an area of 20 μm in diameter. Although few, hardly visible in FIG. 4A, large agglomerates (FIG. 3G and H) would represent a disproportionate mass of total particles detected in the liver section. To represent the percent of total particle mass, the distribution of the total FNDP-(NV) positive area is presented in FIG. 4B. By area, the modal diameter of particle agglomeration is roughly 14 μm. In one animal, large agglomerations 40-100 μm in diameter can be found in the venous system that account for as much as 20% of the FNDP-(NV) positive area, though agglomerates of this size were not found in the second animal.

### Confocal Fluorescent Microscopy (CFM) of Preserved Liver Slices

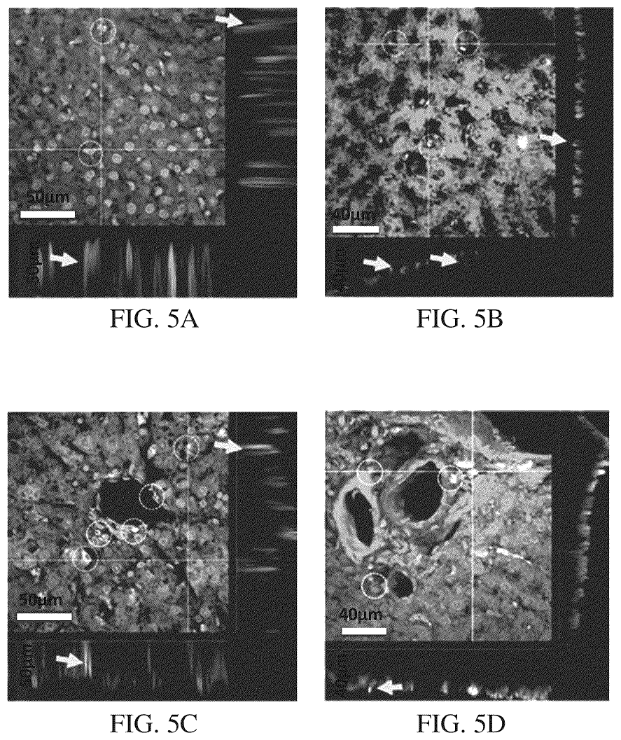

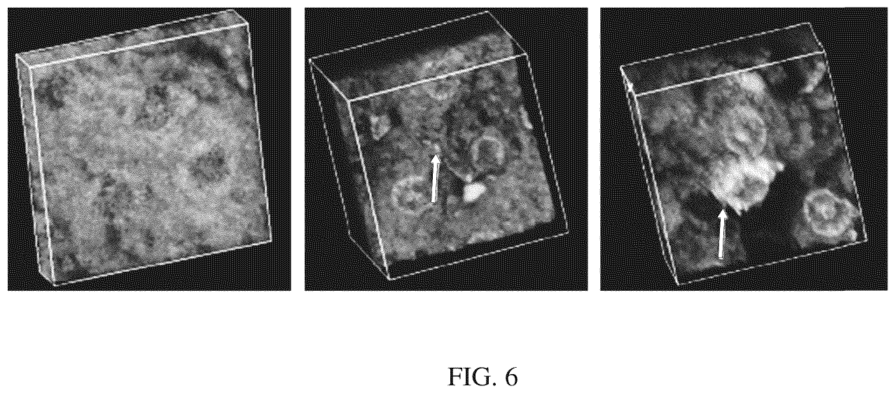

FIGS. 5A-5D presents four different topographical segments studied by CFM on liver sections (10-50 μm). In FIG. 5A several peri-nuclear particles agglomerates of about 5-10 μm are visible (yellow circles and arrows), yet definite intra-cellular location cannot be established. FIG. 5B presents intercellular spaces likely representing portal sinusoid of which some contain large agglomerates of FNDP-(NV) at 10-30 μm (yellow circles and arrows). The intense red coloring suggests location sufficiently remote from the internal milieu of the parenchyma cells (stained green), though some yellow, indicating potential for at least partial internalization, is present as well. FIGS. 5C and 5D present several non-parenchyma structures (surrounded by parenchyma cells) such as venous, arterial, portal vein and likely a bile duct. In FIGS. 5C and 5D several small particle agglomerates (white circles) are located in the sub-endothelial zone of the vessel intima while some agglomerates residing inside parenchymal cell (yellow circles) are also noted.

FIG. 6 illustrates confocal 3D reconstruction of hepatocytes with differing amount of incorporated FNDP-(NV). Two areas are presented which differ in the mass of particles; the cells in the center panel acquired few while the cells in the right panel appear to have been amassed very large particles agglomerates. The left panel represents the vehicle treated rats; no particles have been identified there. In all of the examples provided, the nucleus and nucleoli of these cells present same and normal phenotype.

### Kinetic of FNDP-(NV) Uptake Into Cultured HUVEC and HepG-2 Cells

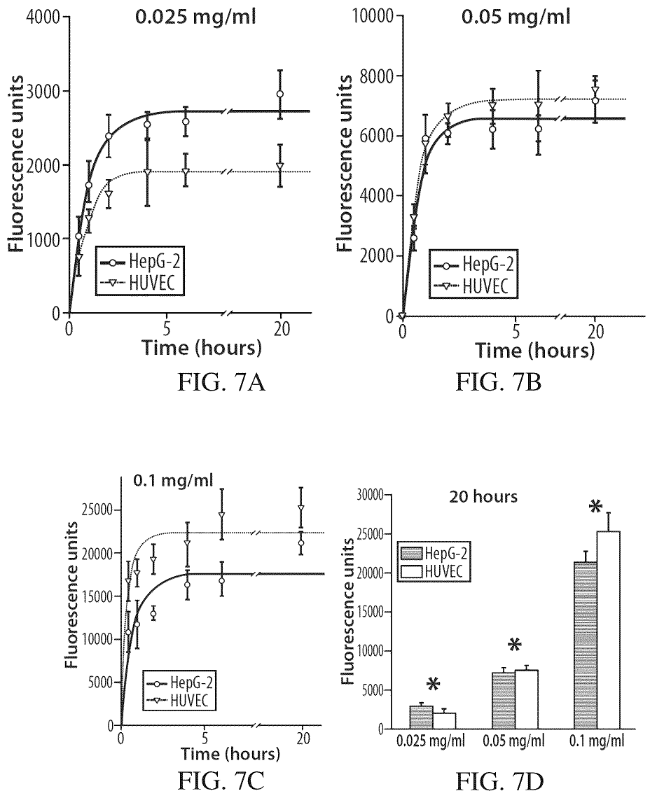

FIGS. 7A-7D depict the kinetics of FNDP-(NV) uptake into HUVEC and HEPG-2 cells under various concentration and time course conditions. FIGS. 7A-7C represents the time course at three different exposure levels of FNDP-(NV). Each of the exposed dose demonstrated same pattern of rapid uptake of particles into the cell body. The rapid uptake phase is attenuated within 1-2 hours reaching a plateau proportional to the amount of FNDP-(NV) exposure. FIG. 7D represents the quantitative accumulation of FNDP-(NV) monitored by NIR fluorescence for each of the cell lines at the three concentrations of FNDP-(NV). The difference in total accumulated FNDP-(NV) is statistically significant between exposure levels, but is similar between cell lines.

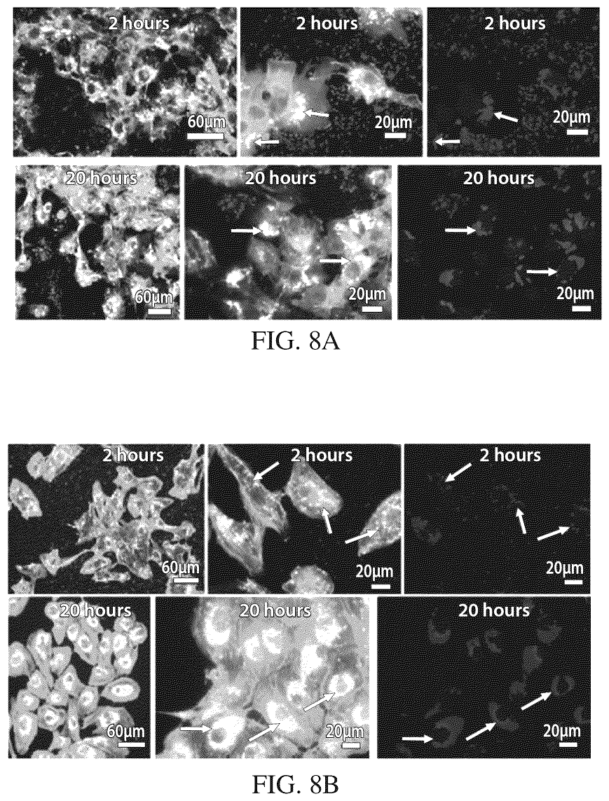

FIGS. 8A and 8B are FM micrographs of FNDP-(NV) accumulation at an early and late stage of the in vitro experiment. The early phases (up to 2 hour) demonstrate particles largely in the cytoplasm while the terminal time point (20 hours) reveals heavy agglomeration in the form of a peri-nuclear corona. Such a pattern was also documented in the preserved liver slices (12 weeks post exposure), as seen in FIG. 6C.

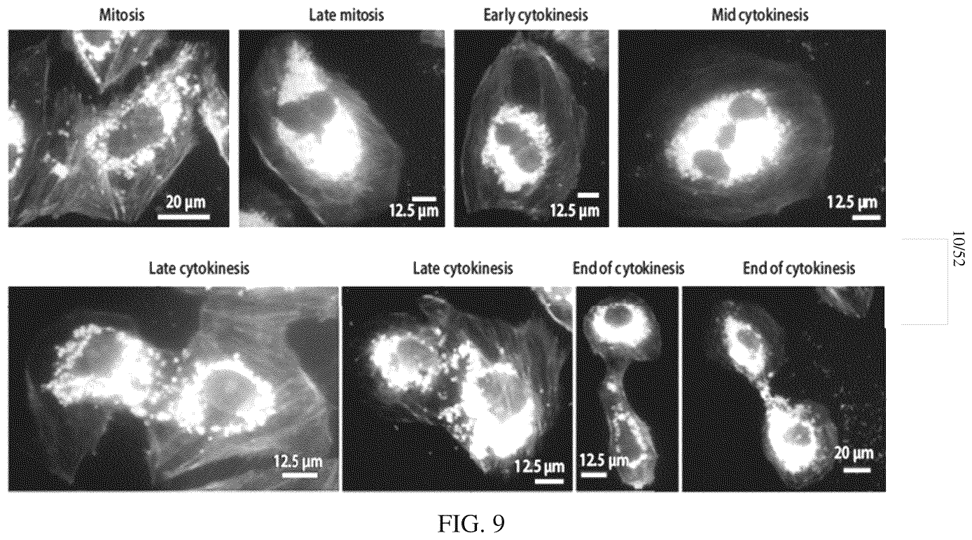

The images of HUVEC in the early mitosis through the end of cytokinesis are presented in FIG. 9A for cells treated with FNDP-(NV) for 20 hours, and in FIG. 9B for untreated, control cells. All treated cells display heavy peri-nuclear FNDP-(NV) accumulation, including in late stage cytokinesis and cell separation. Similar observation has been made in the control group.

### Discussion

The gross distribution of a high dose (60 mg/Kg) of FNDP-(NV) infused to intact rats were characterized and their dispositions were followed acutely, (90 min), sub-acute (5 or 14 days), and long-term (12 weeks) post FNDP-(NV) exposure. Analysis of particle distribution across 6 organs (liver, spleen, lung, kidney, heart and brain) confirmed the liver as the primary repository organ for these particles. Organ histology evaluation did not reveal any FNDP-(NV) related gross or histopathology adverse effects. The lack of adverse effects related to FNDP-(NV) is in accord with reported normal liver function tests.

As described above, the persistence of large numbers of FNDP-(NV) particles in the liver would generally raise concerns about potential negative impacts, especially in the context of long-term residency, possibly indefinite presence. Such prospect might raise regulatory hurdles and potentially impact the GLP (Good Laboratory Practice) pre-clinical development for human use. While a certified pathology report confirmed the lack of histopathological findings, the present investigation was aimed at addressing three primary objectives: 1. Comprehensive survey of FNDP-(NV) distribution in the various liver cells, including intra-cellular location in hepatocytes. 2. Localization of FNDP-(NV) in the microvascular system of the hepatic lobule. 3. Explore the kinetics of FNDP-(NV) particle uptake into cultured liver cells and their intracellular distribution using surrogate (proxy) cell cultures such as HUVEC (endothelium) and HepG-2 (human liver carcinoma) cells.

The primary outcomes of this study include: 1. revealing the unique pattern of spatial distribution of FNDP-(NV) in the hepatic lobules, including parenchymal cells (hepatocytes), non-parenchymal cells (vascular endothelium and adventitia cells) and the venous supply (portal vein) and drainage (central vein) system. 2. Demonstration of intracellular uptake and compartmentalization of FNDP-(NV) in liver cells in vivo and in vitro. 3. Affirmation of the preservation of normal macro and micro morphological phenotypes of liver cells including cells with large coronas of particles in the peri-nuclear space. 4. Preservation of viable cytokinesis processes, from late mitosis to completion of cytokinesis to cell replication including cells with extensive peri-nuclear coronas.

The distribution of FNDP-(NV) across the complete “panoramic” display (FIGS. 3A-3B) revealed a repetitive pattern prevalent in the hepatic “hexagonal” lobules at large (see FIGS. 3D, 3E, and 3F). Particle aggregates were more prevalent at the periphery of the hepatic lobule, surrounding the ‘portal triads’ (PT), yet rather scarce in regions more proximal in the vicinity of the CV. While the mechanism(s) for such distribution are currently not clear, it is hypothesized that this kind of spatial distribution of FNDP-(NV) across the hepatic lobule could be the result of several converging factors.

First, FNDP-(NV) delivery via the PVs often presented aggregated particles at sizes that could barely fit the sinusoid diameter or even exceed it. As shown in FIG. 4A, 30-40% of the detected FNDP-(NV) agglomerates were more than 7 μm, making them, without wishing to be bound by theory, prone to mechanical capture at the more proximal part of the sinusoids. While this does not account for the majority of FNDP-(NV) positive regions by number, these particles account for 75-85% of the total FNDP-(NV) positive area (FIG. 4B), which may account for the strong fluorescence bias within the lobule, despite the significant number of smaller aggregates (individual or limited replicates) which could travel further down the sinusoid, transverse the sinusoids and recycle into the systemic circulation. The presence of small particles at the entry port of the sinusoid into the central vein (CV) supports this possibility (see FIGS. 3D and 3G).

Second, Kupffer cells that serve the scavenging function of the liver (the Reticular-Endothelial System, RES) are generally abundant in the sinusoids and more so at the proximal zone of the sinusoids exiting from the PV. These macrophage-like cells rapidly scavenge particles with preferential kinetics for the larger over smaller particles, which in the case of the FNDP-(NV) will augment their deposition more proximal to the PV over the CV zone, as demonstrated by the data.

Third, the terminal zone of the sinusoid/venule is generally more ‘spacious’ than the port of exit of the sinusoid from the PV. Such anatomy could support hemodynamic conditions, which facilitate clearance of particles into the CV, and further down into the systemic circulation, thereby contributing to the relative paucity of particles in vicinity of the CV.

Fourth, the venous microcirculatory system is a critical element in securing the hepatic lobule's most delicate biochemical functions. The data described herein clearly indicate the presence of large particle aggregates in the PV and possibly CV along with enhanced presence in the outer circumference of the hepatic lobules (peri PT), and scant but notable small particles throughout the lobule (see FIG. 3E). Particles within these spaces could interfere with the delicate balance of blood flow in the sinusoids, causing hemodynamic disturbances (e.g., turbulence flow) and congestions that obstructs the flow. Disruption of flow could bear on oxygen delivery as well as distribution of nutrients to the parenchymal cells, thereby negatively affecting synthetic and catabolic functions of the liver. While micro-hemodynamic disturbances in the sinusoids cannot directly be ruled out, detailed histological analysis (Supplementary Materials) failed to observe areas of blood congestions (due to partial blood flow blockage), thrombosis (due to stasis), or ischemic consequences at a microscopic level.

Nevertheless, the topographical inhomogeneity of FNDP-(NV) distribution could still carry physiological implications by virtue of particles mass or size, intra-cellular location localization and micro-hemodyanmics factors not yet matured (at the time of the study termination) to manifest aberrant consequences on the anatomy and physiology of the hepatic unit at large. The peripheral zone of the hepatic lobules, where larger aggregates of particles were most prevalent (see zone 1 in FIG. 3H), is the locale for many important and critical biochemical and cell survival functions in the liver (e.g., fatty acids oxidation, gluconeogenesis, bile production, xenobiotics metabolism and regenerative cells replenishment).

Support for the likely preservation of liver morphology at the micro-environment is presented in FIGS. 6A and 6B. The topological survey across the panoramic field of the whole liver surface suggests that percent of particles and the area that they occupy are only a small (or moderate) fraction of the total. Since the data presented in this manuscript evaluated a situation generated 12 weeks earlier, acute post FNDP-(NV) exposure cannot be rejected.

Lastly, cell culture studies were performed to gain insights on the direct interactions of liver cells with FNDP-(NV) in an isolated system to explore the kinetic of particle internalization, compartmentalization and viability of cytokinesis capability in the presence of particles. The two different cell types, used as surrogate for the respective human hepatocytes and endothelial cells, indicated rapid initial uptake of FNDP-(NV) into the cells in time-and concentration-dependent, manner. This in vitro study supports the in vivo observations of intracellular uptake of FNDP-(NV) into non-scavenging liver cells (hepatocytes).

### Summary

In this work, the interactions of FNDP-(NV)-Z-700/800 nm with liver cells in vitro and in vivo were studied. These studies addressed the scale and extent of FNDP-(NV) deposition in terms of their cellular and sub-cellular resolution, their presence in parenchymal and non-parenchymal cells, as well as in the micro-circulation. In vivo data were complemented by studies conducted in vitro (HUVEC, HepG-2 cells), where direct kinetic studies of particle uptake and assembly in these surrogate liver cells supported the results obtained from whole animal exposure study. Taken together, the data described above strongly suggests liver bio-compatibility of the FNDP-(NV), as no aberrant consequences could be identified in terms of preservation of cellular phenotypes, cytoskeletal, nuclear structure, as well as unabated cytokinesis and cell replication. As such, FNDP-(NV) could potentially be well tolerated by humans exposed FNDP-(NV) by intravenous route of exposure.

### Example 2

The following example describes cellular and biochemical functions in cultured Human Umbilical Endothelial cells (HUVEC) and human hepatic cancer cell line (HepG-2) exposed to FDP-NV-700/800 in vitro at exposure levels within the pharmacokinetics (Cmax and the nadir) reported in vivo.

Nanomedicine is a fast-growing medical discipline featuring intense pre-clinical research and emerging clinical exploratory studies as evident by over 25,000 articles listed in PubMed over the past 10 years. Nanomedicine offers a ‘third leg’ of pharmaceutical technology above and beyond synthetic organic molecules and engineered biologicals. Nanomedicine builds on diverse materials co-junctional to additives that aim to direct biologically active nanoparticles to specific cells, organs, or pathological processes.

Of major contemporary interest are particles engineered to emit near infrared (NIR) light in response to an electromagnetic stimulus (excitation light) that generates fluorescence either due to innate properties (e.g., “Color Centers”) or coatings with organic fluorescent additives. The ability to emit in the NIR opens the possibility for imaging of bodily structures per se or as adjunct to state-of-the art imaging technologies (e.g., MRI/magnetic resonance imaging, ultrasound) along with targeted delivery of therapeutic agents.

Of particular interest are diamond particles, such as nanodiamond particles or fluorescent diamond particles, carrying nitrogen-vacancies (FDP-NV-) that enable the particles to become fluorescent upon excitation at 580-620 nm, resulting in near infrared (NIR) emission in the peak range of 720-740 nm. The NIR light emission of such particles displays exceptional stability, negligible interference by biological elements such as water and oxyhemoglobin. Furthermore, surfaces of these particles can be functionalized with a variety of chemical groups (carboxyls, amines, etc.) that provide diverse linkages opportunities, from small organic molecules, to polymers, proteins, and nucleic acids.

It has been discovered within the context of this disclosure a bioengineered fluorescent diamond particles-NV-Z˜700/800 nm (FDP-NV) conjugated with snake venom disintegrin, bitistatin (Bit), and it has been shown (in vitro and ex vivo) that FDP-NV˜800 nm/Bit binds specifically to the platelet fibrinogen receptor αIIbβ3 integrin. Subsequently, in vivo studies have demonstrated the binding of FDP-NV-Bit to acutely generated (iatrogenic) blood clots in rat carotid arteries. Taken together, FDP-NV˜800 nm/Bit demonstrated targeted homing in vivo and hence showed the potential to serve as a diagnostic tool for high-risk vascular blood clots.

The studies were followed by 3 safety and biocompatibilities studies, where a high dose (60 mg/Kg, delivered as a single intravenous bolus) of FDP-NV˜800 nm (FDP-NV) blocked by BSA was infused to intact rats to establish the pharmacokinetic profile, organ distribution and to assess a comprehensive panel of hematologic, metabolic and biochemical safety biomarkers. In these studies, it was found that within the 5 days to 12 weeks follow up periods, FDP-NV primarily distributed to the liver and spleen, and that virtually none were found in the lung, heart, and kidney. Furthermore, no specific histopathological observations related to the FDP-NV particles were observed. However, no study so far addressed possible acute safety or toxicological consequences in endothelial or hepatic parenchyma cells exposed to FDP-NV-700/800 nm.

In the present example, the search for possible direct FDP-NV-800 nm related toxicological effects were studied using two different cell-types, HUVEC and HepG-2. These cells were chosen since endothelial cells are the first line of exposure to FDP-NV when infused into the systemic circulation (as per the intended clinical indication), while hepatocytes are the primary repository of circulating FDP-NV. FDP-NV exposure levels were selected according to the acute pharmacokinetic levels observed in vivo, including the maximal blood levels and its nadir at 90 minutes post-exposure. Considering that acute biocompatibility studies with FDP-NV-800 nm have yet to be reported in the published literature, the studies presented here provide new information and insights into the acute biocompatibility of FDP-NV-700/800 nm in support of the intended clinical development in humans.

The data overall support reasonable biocompatibility of FDP-NV-700/800 nm with respect to short term proliferation at Cmax exposure in cultured HUVEC. HepG-2 have not been affected at the same exposure and time.

### Methods

Diverse cellular and biochemical functions were monitored, which in summation provide insights on the cells'integrity and vital functions. Cell proliferation, migration, and regeneration were assessed by quantitative microscopy. Mitochondrial (oxidative) functions were tested by MTT redox reaction and cytosolic esterase activity studied by calcein AM assay. ER-stress biomarkers were examined by chaperons CHOP and BiP and apoptosis by caspase-3 activation using Western blot (WB). MAPK Erk1/2 signaling was assessed by detection of the phosphorylated from of the protein (P-Erk 1/2) and its translocation into the cell nucleus.

### Results

Exposure of HUVEC cells to 100 μg/mL FDP-NV (Cmax) suggested potential adverse effects on cell proliferation, cytosolic esterase activity, and oxidative functions. Cell signaling (MAPK Erk1/2) and ER-stress biomarkers remained intact as did the activation of the pro-apoptotic pathway (caspase 3 activation). With a similar exposure and time frame, no aberrant tests have been observed in HepG-2 cells, which demonstrated resilience in some studies at some levels of exposure.

### Material and Methods

### Preparation of Nanoparticles

Carboxyl-functionalized FDP-NV˜800 nm (FDP-NV) were purchased from ADAMAS Nanotechnologies (Raleigh, NC, USA). FDP-NV were sanitized by suspension in 70% ethanol for 15 min at room temperature (RT) followed by centrifugation for 7 min at 2900×g at RT to isolate the particles. Passive blocking of potential non-specific protein binding sites on the particles was performed by incubation with PBS (phosphate buffered saline, pH=7.4, ThermoFisher Sci., Waltham, MA, USA), containing 3% BSA (bovine serum albumin, Sigma, St Louis, MO, USA) at 37° C. for 1 hour. FDP-NV-BSA were isolated by centrifugation as described above and particles were stored as a stock solution in PBS at 1 mg/mL in 4° C.17

### Analysis of Z-Average and ζ-Potential of FDP-NV-700/800 nm in Different Dispersants

Particles blocked with BSA (FDP-NV-BSA) or ‘naïve’ (FDP-NV-COOH, pre-BSA blocking), were suspended in deionized (DI) water, PBS, or culture media according to the various protocols used in the cell experiments (vide infra). HepG-2 (human liver hepatocellular carcinoma) cells were cultured in Eagle's Minimum Essential Medium (EMEM, ThermoFisher Sci.), supplemented with 10% fetal bovine serum (FBS) (ThermoFisher Sci.) and penicillin/streptomycin (ThermoFisher Sci.), HUVEC were cultured in EGM-2MV media (Lonza, Basel, Switzerland). Particles were suspended in each culture media as dispersant at a density of 0.5 mg/mL and applied into dual-purpose capillary cuvettes (1 mL total volume). Samples were tested in a Zetasizer Ver. 7.11 (Malvern Panalytical Ltd., Malvern, UK).

### Cell Counting Assay

Cell counting was performed. The HepG-2 cell line was purchased from American Type Culture Collection (ATTC) (Manassas, VA, USA). Primary HUVEC were purchased from Lonza and used for experiments between the 5th-8th passages.

Cells maintained (37° C. at 5% CO2 atmosphere) in their respective culture media as described above. HepG-2 and HUVEC were ‘seeded’ in 96-well plates (2×103 per well in 100 μL medium) and treated or not with FDP-NV-BSA for 24 hours. In each experiment, vincristine (50 μg/mL), a cell-cycle proliferation inhibitor, was added as a positive control. At 24 hrs, the medium was removed, the cells were fixed with 4% paraformaldehyde (PFA, ThermoFisher Sci.) and the nuclei were stained using DAPI (4′,6-diamino-2-phenylindole, dihydrochloride, ThermoFisher Sci.). The plates were analyzed in a fluorescence microscope (Olympus IX81, Olympus, Tokyo, Japan) by imaging 7 observation fields for each well using 100× magnification and DAPI (blue filter) for nuclei visualization, and TRITC (red filter) for FNDP-NV-BSA visualization. The number of viable cells in each field was determined by analysis of DAPI stained nuclei using ImageJ software (National Institutes of Health, Bethesda, MD, USA) with a digitally set-up cell counter.

### Cells Metabolic Activity Monitored by MTT Assay

The MTT assay was performed as a colorimetric assay using the Cell Proliferation Assay Kit (ThermoFisher Sci.), composed of component A (3-(4,5-dimethylthiazol-2-yl)-2,5-diphenyltetrazolium bromide (MTT)) and component B (SDS (sodium dodecyl sulfate)) according to manufacturer's protocol. Briefly, HepG-2 cells and HUVEC were seeded in 96-well plates at a density of 1×104 cells per well in media described above for each cell type. Cells were treated or not with FDP-NV-BSA or vincristine (50 μg/mL) for 24 hours. Media (100 μL) were changed to phenol red-free DMEM (Dulbecco's Modified Eagle Medium) (ThermoFisher Sci.), containing MTT component A. Plates were incubated for 4 hours, and cells lysed by adding equal volumes of 10% SDS (kit component B). Plates were incubated overnight and read using an ELISA plate reader ELx800 (BioTek, Winooski, VT, USA) at 562 nm wavelength.

Calcein AM Cell Cytosolic Esterase Assay Seeding and treatment of HepG-2 cells and HUVEC were performed as described above for the MTT assay. Cells were treated with 5 μg/mL calcein AM (ThermoFisher Sci.) in serum-free media and incubated for 30 min in 37° C. Plates were read using a florescence microplate reader FLx800 (BioTek) with 485 nm Excitation and 530 emission wavelengths.
“wound Healing” (WH) In Vitro Assay

HUVEC were seeded in 12-well plates and maintained for 1-2 days until 80-90% confluency and treated or not with FDP-NV-BSA for 24 hrs. Confluent HUVEC cells (monolayer) were subjected to a ‘gentle scrape’ across the plate using a plastic spatula tip, resulting in a gap area (devoid of cells) of approx. 1 mm width. Cells treated with FNDP-NV-BSA were stimulated for 24 hrs migration time by replacing the media to those containing 2% FBS. Control cells (non-exposed to FNDP-NV-BSA) were divided for positive stimulated by 2% FBS, and negative where stimulator of migration was minimalized to 0.1% FBS (HUVEC are sensitive for complete removal of FBS and detach from the surface). Cells were fixed with 4% PFA and stained with DAPI, as described above. Imaging of scratches was performed in a fluorescence microscope (Olympus IX81) at 20× magnification and DAPI (blue filter) for nuclei visualization and TRITC (red filter) for FNBDP-NV visualization. Control plates included confluent cells subjected to the same scratch immediately before PFA exposure. The migration index was estimated by measurement of total surface area cell-free region of the images, using ImageJ software.

### Cell Signaling Assay Represented by Phosphorylation of MAPK Erk1/2

HepG-2 cells and HUVEC were cultured in 6 cm diameter Petri dishes to 90% confluency and treated or not with FDP-NV-BSA (density 0.1 mg/mL), as described above. Cells were serum-starved for 24 hours and then stimulated with 2% FBS for 0, 10, and 20 minutes. Cells were lysed in ice-cold RIPA (Radioimmunoprecipitation assay) buffer (Teknova Inc., Hollister, CA, USA), containing a ‘cocktail’ of protease inhibitors (Sigma Inc.) and the “Halt” phosphatase inhibitor cocktail (ThermoFisher Sci.).

Protein lysate (20 μg) was applied on SDS-PAGE (sodium dodecyl sulfate, polyacrylamide gel electrophoresis) using Mini-PROTEAN precast gradient (4-20%) gels (Bio-Rad Inc., Hercules, CA, USA), and transferred into PVDF (Polyvinylidene difluoride) membranes (Sigma Inc.) using a semi-dry blotting system (Bio-Rad Inc.).

The presence of phospho-and total-Erk1/2 (after membrane ‘stripping’) was detected using polyclonal antibodies (Cell Signaling Techn., Danvers, MA, USA). Visualization of the protein bands on the membrane was performed using a C-DiGit Blot Scanner (LI-COR Biosci., Lincoln, NE, USA). The intensity of the bands was quantified using UN-Scan-It software (Silk Scientific Corp., Orem, UT, USA) for calculation of the ratio of phosphor-Erk1/2 to total-Erk1/2.

### Nuclear Translocation of Phospho-Erk1/2

Fractionation of cell lysates. HepG-2 cells and HUVEC were grown in 6 cm diameter dishes, treated or not with FDP-NV (0.1 mg/mL), and ‘starved’ under the same conditions as described above for MAPK Erk1/2 cell signaling. Cells were stimulated by exposure to 250 nM TPA (Tetradecanoyl phorbol acetate, Sigma Inc.) for 15 minutes. Fractionation of the cells was performed using Subcellular Protein Fractionation Kit for Cultured Cells (ThermoFisher Sci.) according to the protocol provided by the manufacturer. Cytoplasmic and nuclear extracts of fractions were analyzed by WB using phospho-and total-MAPK Erk1/2 as described above. Verification of cytoplasmic and nuclear fractions was performed by WB analysis using, an anti-Mek polyclonal antibody and an anti-HDAC1 polyclonal antibody (Cell Signaling Techn.), respectively.

### Immunocytochemistry for the Detection of Phospho-MAPK Erk1/2 in Cytoplasm and Nucleus

Immunostaining of cells cultured in 8-wells glass chamber slides was performed as described previously. 24 Cells were treated or not with 0.1 mg/mL of FDP-NV-BSA in the presence or absence of TPA (see above). A Polyclonal anti-phospho-Erk1/2 antibody (Cell Sign. Techn.) was used in conjunction with a FITC-tagged goat anti-rabbit IgG (Vector Labs Inc., Burlingame CA, USA). Slides were analyzed in a fluorescence microscope (Olympus IX81) using 400× magnification with an oil objective and FITC (green filter) to detect phospho-MAPK Erk1/2 and TRITC (red filter) to detect FNDP-NV.

### Cell Apoptosis Assay (Caspase-3) and Endoplasmic Reticulum (ER)-Stress Biomarkers

HepG-2 cells and HUVEC were treated (or not) with 0.1 mg/mL FDP-NV-BSA as described above. Treatment with vincristine (200 μg/mL) was used as a positive control for apoptosis, and with tunicamycin (25 μg/mL) as a positive control for ER-stress. A rabbit polyclonal antibody against caspase 3 (Cell Sign. Techn.), which recognizes both the cleaved and the non-cleaved protein, was used for apoptosis detection. Rabbit mAb (clone C50B12) against BiP and mouse mAb (clone L63F7) against Chop (both from Cell Sign. Techn.) were used for the detection of ER-stress. Equal loading of proteins was verified by membrane stripping and re-probing with an anti-actin mouse monoclonal antibody (Sigma Inc.).

### Results

### Characteristic of Physical Properties of FDP-NV˜800 nm Suspended in Various Media

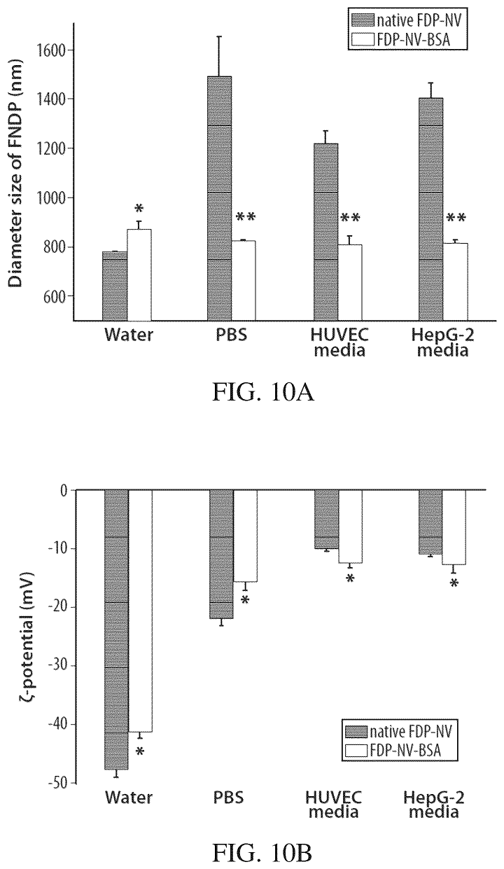

The FDP-NV˜800 nm was suspended in various dispersants known to modify particle diameters (Z-average), and surface ζ-potential. FIG. 10 presents the changes in diameters (Z-average) of FDP-NV-COOH (native particles without passive absorption of BSA) or FDP-NV-BSA suspended in DI water, PBS (pH=7.4), or media used in each of the cell cultures. A substantial and statistically significant increase in the Z-average was observed when FDP-NV-COOH were suspended in PBS; The particle size increased from 778 nm (DI water suspension) to 1488 nm (PBS), 1215 nm (HUVEC media), and 1403 nm (HepG-2 cell media), respectively. Passive absorption of BSA on the surface of the particles (FDP-NV-BSA) neutralized the increases observed in the respective solutions. The ζ-potential was substantially impacted trending to more positive charges to isoelectric (e.g., FIG. 10B). The ζ-potential of FDP-NV˜800 nm-COOH dispersed in DI water was −47.9 mV, which increased to −21.9 mV when the particles were immersed in PBS, −9.9 mV for HUVEC media and −10.9 mV for HepG-2 cell media, respectively. However, unlike the impact on Z-average, passive coating of FDP-NV with BSA had minimal impact on the ζ-potential of FDP-NV-COOH. It is noteworthy that the media used for HUVEC or HepG-2 did not differ in their impact on either the Z average or the ζ-potential.

### Effect of FDP-NV on HUVEC or HepG-2 Cell Proliferation

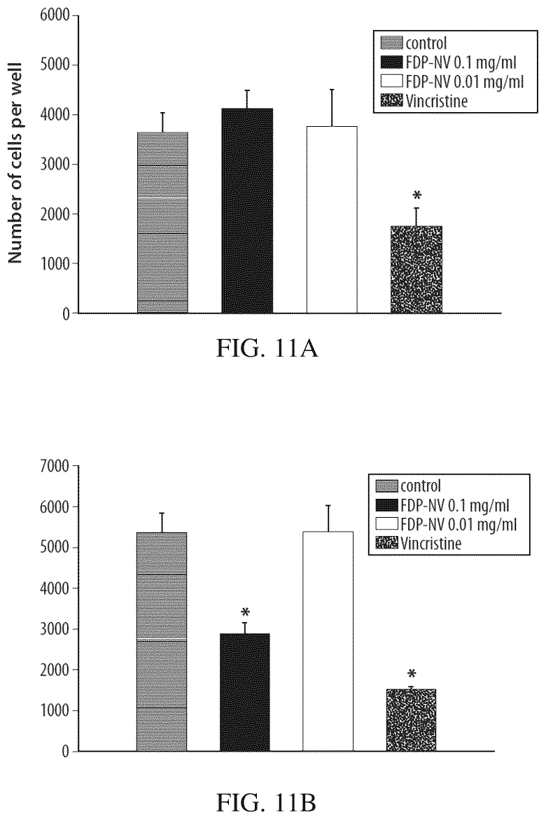

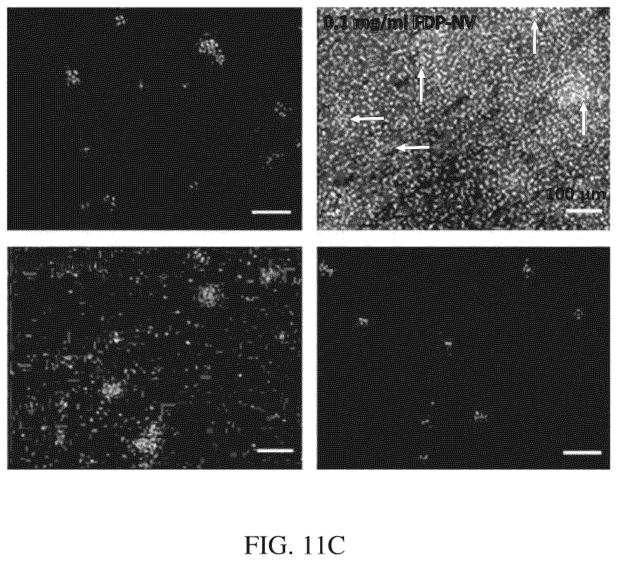

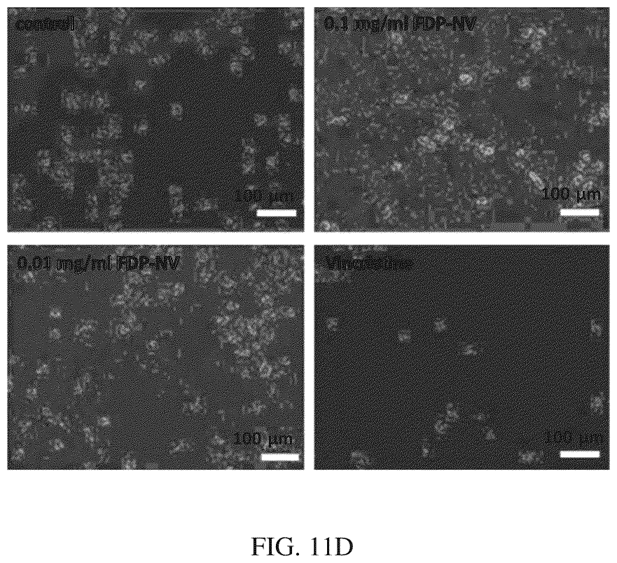

HepG-2 cell line was not impacted by the presence of FDP-NV-BSA (up to 0.1 mg/mL), as inferred from the increase in cell numbers over 24 hours (FIG. 11A). In contrast, HUVEC exposed to a high concentration of FDP-NV (0.1 mg/mL FDP-NV-BSA) showed a statistically significant reduction in the cell number to approximately 60%. Impact following exposure of HUVEC was not observed to a lower concentration ( 1/10th) of particles (FIG. 11). As expected, vincristine suppressed proliferation to 50% and 80% of controls in HepG-2 and HUVEC, respectively. Representative images of cells treated for 24 hours with 0.1 mg/mL FDP-NV-BSA confirmed particle accumulation uptake into the cells and their peri-nuclear agglomeration especially in HUVEC (FIG. 11D). Similarly, but less conspicuously, HepG-2 cells also displayed an accumulation of FDP-NV-BSA in cytoplasm and formation of a perinuclear corona (e.g., FIG. 11C). This observation is in accord with recently reported studies in both cell types.

Effect of FDP-NV-BSA on the redox intensity in cultured HUVEC or HepG-2 cells.

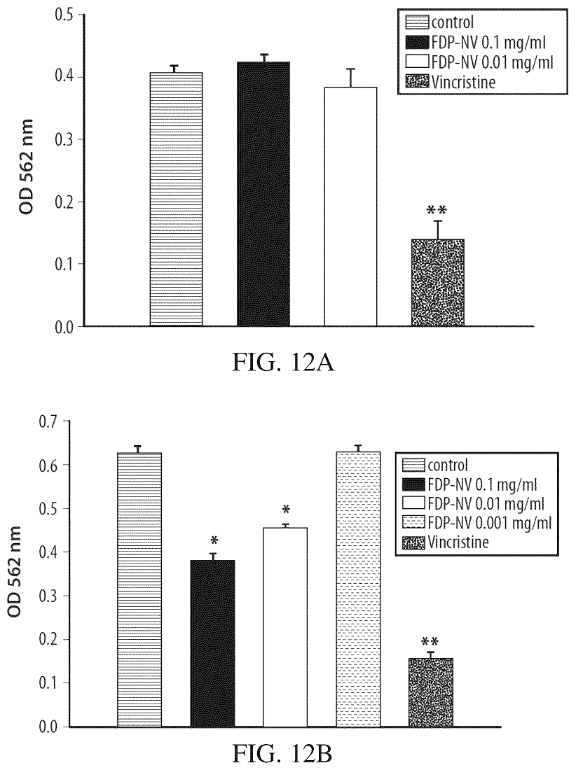

The redox state of cultured HepG-2 cells, as assessed by MTT, was resilient to the presence of FDP-NV-BSA at some concentrations including Cmax (0.1 mg/mL, FIG. 12A). In contrast, HUVEC demonstrated a diminished oxidative capacity at exposure levels within the Cmax and nadir (0.01 mg/mL) of the pharmacokinetic blood levels.

However, at lower tested concentration of FDP-NV-BSA, (0.001 mg/mL), MTT activity as indistinguishable from that of the untreated controls (FIG. 12B). The positive control, vincristine, decreased redox activity to ˜25-30% of normal controls for both cell types.

### Effect of FDP-NV-BSA on HUVEC or HepG-2 Cell Cytosolic Esterase Activity

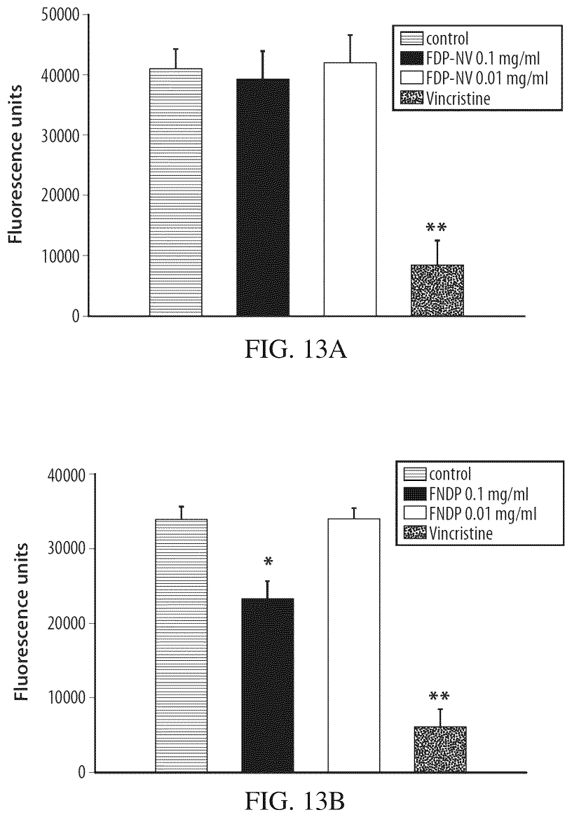

The calcein AM assay provided information on non-specific esterase activities in the cytosol. FIG. 13 shows no deviation of this test in HepG-2 cells (FIG. 13A), while HUVEC (FIG. 13A) showed a ˜30% reduction at a concentration of 0.1 mg/mL FDP-NV and no interference at the nadir level of exposure (0.01 mg/mL).

Effect of FDP-NV-BSA on HUVEC Migration Stimulated by FBS in a “scratch” Injury Model In Vitro

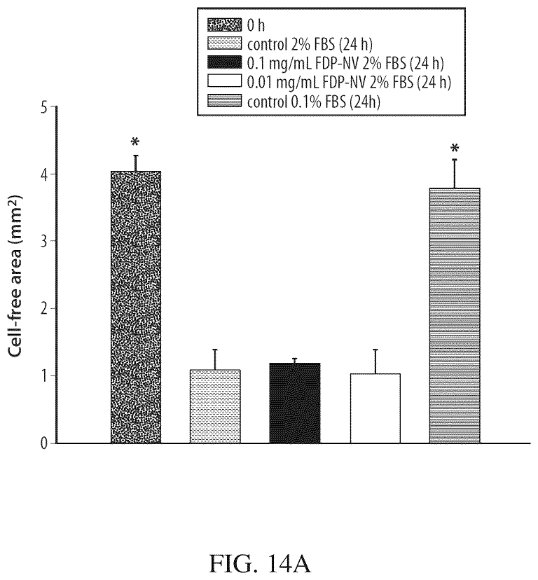

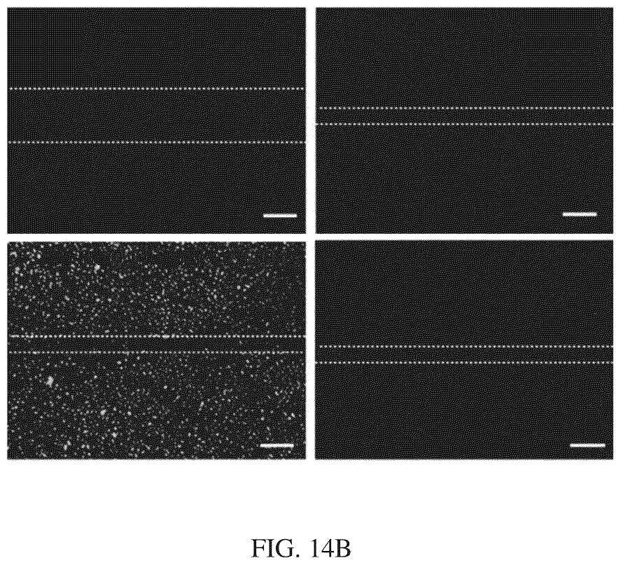

The effects of FDP-NV-BSA on cell migration were investigated in an in vitro model of ‘wound healing’ (“scratch assay”, FIG. 14). This assay was applied only for HUVEC since the pattern of growth of HepG-2 (forming clusters of colonies) was not suitable for this test. Quantification of cell migration across an artificially generated cell-free region (area of scratch) revealed no difference between control, untreated cells and cells exposed to FDP-NV-BSA. HUVEC treated with 2% FBS migrated readily even when exposed to a high concentration of the particles (0.1 mg/mL, FIG. 14A).

Interestingly, the fluorescence microscopic images revealed a visually similar particle burden of internalized FDP-NV-BSA (overlapping blue and red colors, shown in different shades of gray) in the active cells (migrating into the “scratch zone”) and in “stationary” cells located outside the scratch zone (FIG. 14B).

### Effect of FDP-NV-BSA on the Activation of MAPK Erk1/2 in HUVEC and HepG-2 Cells

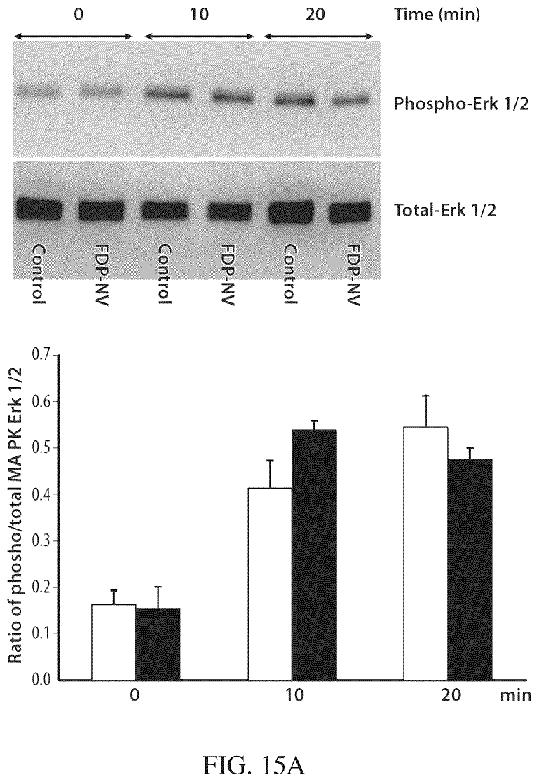

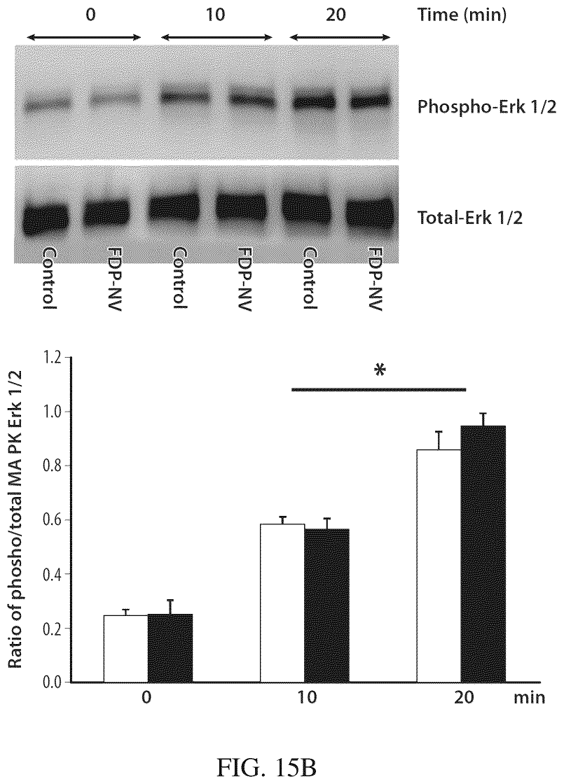

FIG. 15 shows no significant difference in the FBS-induced activation of MAPK Erk1/2 between HUVEC and HepG-2 cells exposed or not to 0.1 mg/μL FDP-NV-BSA two time points (10- and 20-min) post stimulation. Interestingly, HepG-2 cells reached the plateau of FBS stimulation in 10 min (FIG. 15A), whereas HUVEC reached maximal phosphorylation of MAPK Erk1/2 in 20 min (FIG. 15B).

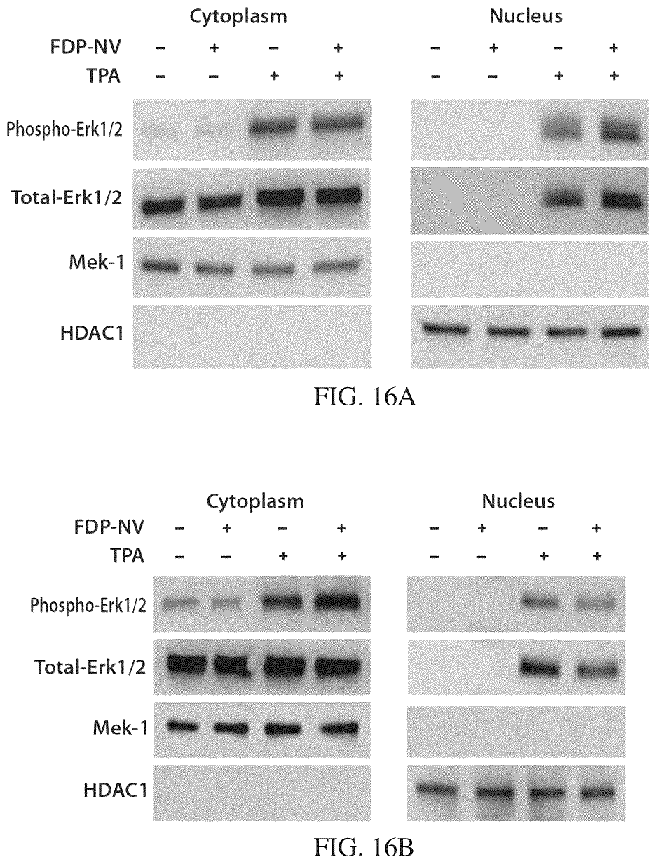

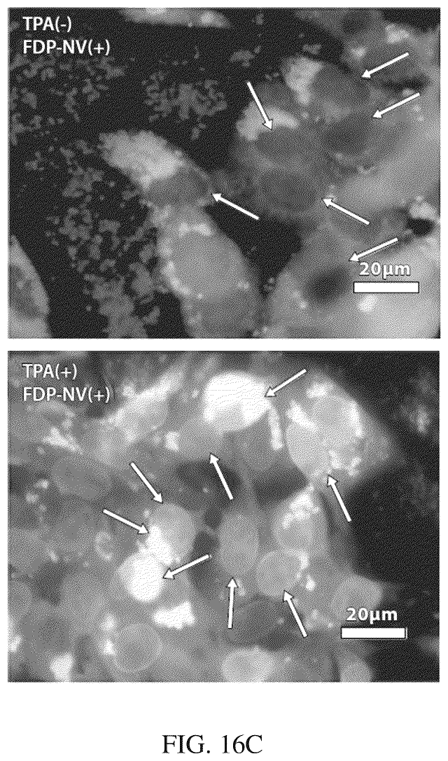

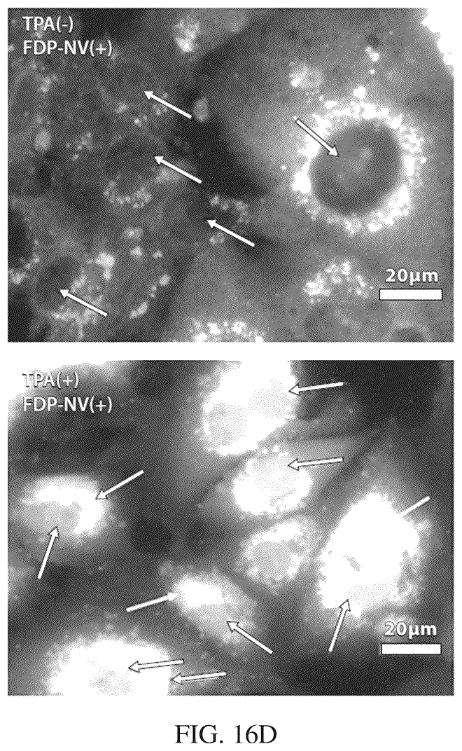

Translocation of proteins from cytosol to nucleus is one of the paradigms that may be affected by intense peri-nuclear accumulation of FNDP-NV. Therefore, translocation of phospho-MAPK Erk 1/2 to nucleus was tested using an applied stimulator of this process, TPA. For this, the cells were fractionated and assessed phospho-and total-MAPK Erk1/2 in the cytoplasmic and nuclear fractions by WB (FIGS. 16A-16B) and by fluorescence microscopy (FIGS. 16C-16D). HepG-2 cells (FIG. 16A) and HUVEC (FIG. 16B) showed no difference between FND-NV-BSA exposed and control (no exposure) cells in the amount of phosphor/total MAPK Erk1/2 in their respective nuclei or cytoplasm. It should be noted that exposure to TPA potentiated the internalization and perinuclear accumulation of FDP-NV-BSA, which could be observed in the fluorescence microscopic images as intensive, yellow color (overlap of red and green (FIGS. 16C-16D).

### Effect of FDP-NV-BSA on the Induction of Apoptosis and ER-Stress

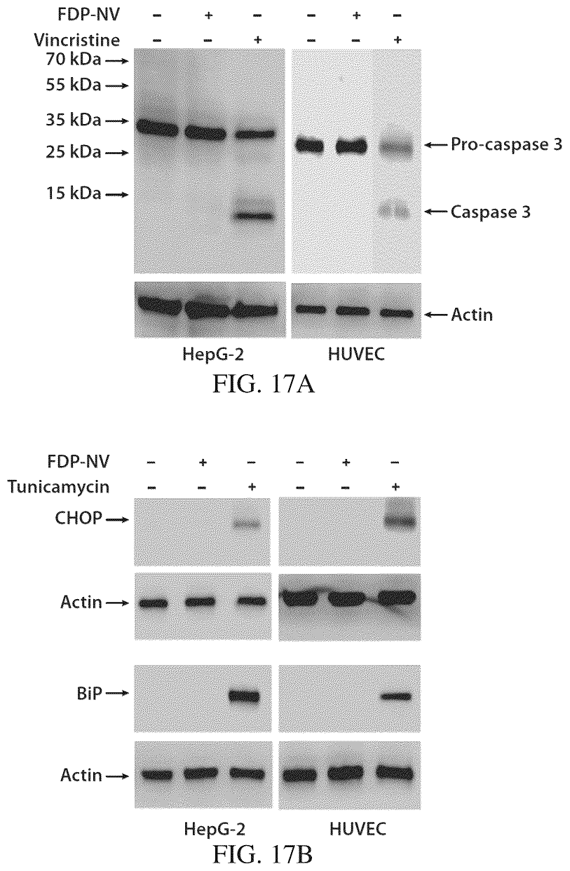

The internalization of FDP-NV-BSA into the cells'cytoplasm and perinuclear accumulation may suggest a possible interference in essential traffic between the nucleus and cytosol, leading to stress conditions as manifested by activation of apoptosis or ER-stress. Therefore, both HepG-2 cells and HUVEC biomarkers were evaluated for stress conditions, such as caspase 3 activation and expression chaperon proteins, CHOP and BiP, using WB (FIG. 17). Exposure to FDP-NV-BSA (at 0.1 mg/mL) did not yield activation of caspase 3 in either of the cells in contrast to vincristine (positive control, FIG. 17A). Strong perinuclear accumulation of FDP-NV appears to persist without consequences within the experimental time. The expression of two chaperon proteins, CHOP and BiP, was also not impacted by the presence of the FDP-NV. Furthermore, there was no apparent difference between HepG-2 and HUVEC (FIG. 17B). Both types of cells were sensitive to tunicamycin, which served as standard control for ER-stress protein activation.

### Discussion

The present set of experiments were a aimed at exploring the safety of FDP-NV (800 nm) and constitute part of the pre-clinical evaluation of these particles, before we commence Good Laboratory Practice (GLP) and Good Manufacturing Practice (GMP) for phase I clinical studies. The safety and tolerability of FDP-NV-800 nm administered intravenously at a relatively high dose to intact rats, high biocompatibility was seen as inferred from the lack of morbidity and mortality monitored over 5 days, 14 days or 12 weeks post exposure. No aberrant hematological and biochemical functions, including blood cells number and differential, or histopathological observations in liver, kidney and lung were detected in all of these studies. Furthermore, the pharmacokinetics ranges following acute infusion of a high dose of FDP-NV-800 nm, indicated rapid clearance from the systemic circulation and fast up-load into the liver and spleen. It is important to note that particles deposited in the liver and spleen were retained over the 12 weeks follow-up.

The present experiments were designed to address potential adverse effects of FDP-NV-800 nm in an acute in vitro setting to gain deeper insights into potential biochemical consequences that could not be discerned by histological and histochemical biomarkers in vivo. Such studies are justified since no record of public data can be found, investigating the same FDP-NV size (˜700/800 nm). Moreover, acute safety biomarkers might not display in the subacute or chronic dosing studies due to compensatory mechanism following exposure.

Adverse interactions of nanodiamond particles with cellular functions have already been reported albeit using different particle sizes, shapes, and adjuvants. These reports stress the importance of probing the effects of FDP-NV˜800 nm on cellular functions, especially of cells that will be exposed to the maximum blood levels (Cmax) during infusion of the particles and shortly thereafter. Naturally, endothelial cells and circulating blood cells are the prime targets for acute, high dose exposure and as are liver cells, which serve as an instant repository of the FDP-NV-700/800 nm. Indeed, pilot studies with FDP-NV˜800 nm using HUVEC and HepG-2 cells revealed uptake of particles into each of these cells'cytoplasm (over 1-2 hrs.) with ultimate peri-nuclear zone assembly to form “coronation” over 20-24 h post-exposure. However, cytogenesis and cell division of these target cells were preserved, suggesting that cell cycle and trafficking in and out of the nucleus remained intact.

The present studies extend observations to probe additional key cellular functions and biochemical processes, including cell proliferation, migration, and signal transduction ER-stress, and apoptosis that are cardinal for cell integrity. The present studies followed pharmacokinetic data obtained after high dose (60 mg/Kg) infusion in the in vivo (rat) experiments. In the studies described in this manuscript, cultured cells were exposed to Cmax levels (immediate post-infusion, 0.1 mg/mL) or nadir (0.01 μg/mL, 90 minutes post infusion) over 24 hrs.

A well-known issue concerning size changes of FDP-NV-COOH upon suspension in solutions containing electrolytes, proteins and various organic additives was addressed. Indeed, monitoring the Z-average and ζ-potential of FDP-NV˜800nm-COOH in DI water (the native product provided by the manufacturer) revealed close similarity with the manufacturer's information (778 nm for Z-average) and −48 mV for ζ-potential. The marked shifts in Z-average generated by dispersing the particles in PBS or culture medium were abrogated in the presence of 3% BSA yet had only a mild (or negligible) impact on ζ-potential shifts (FIGS. 10A-10B). The possible impact of persistent ζ-potential changes on the experimental outcomes remains to be explored in detail.

The cellular effects of nanodiamond particles (NDP) have intensively been investigated in vitro with a variety of cultured cell types, mainly in terms of cell viability, as reported by the MTT assay. In general, NDP are well tolerated by most cell types, when incubated in complete media. The mitochondria-dependent respiratory chain is not affected by NDP even at extremely relatively high concentrations, 1 mg/mL. Capping exposure at the Cmax concentration of 0.1 mg/mL, suggested no interference in the redox state of the HepG-2 cell line (FIG. 11), in line with prior reports on other cancer cell lines. By contrast, a significant inhibitory effect was noted in HUVEC (FIG. 11B), in line with previous data using the immortalized HUVEC-ST cell line. Direct cell counting and the calcein AM assay also suggested interference of the particles with cytosolic esterase activity in HUVEC at 0.1 mg/mL, although, in contrast to MTT, no effect was observed for lower (nadir) levels of 0.01 mg/mL. These data suggest that primary cells, in contrast to a cell line, may be more sensitive to FDP-NV in terms of vital biochemical processes and overall cell functions.

The proliferative cell signaling pathway MAPK Erk 1/2 was not affected by exposure to FDP-NV at the Cmax dose in either cell type (FIG. 15). The extensive peri-nuclear accumulation of particles suggested a potential interference by this “coronation” in the cytosol-nucleus cross trafficking. This possibility was probed by tracking the translocation of phospho-Erk 1/2 into the nucleus. FIG. 16 indicates the presence of P˜phospho-Erk 1/2 in the nucleus following activation of this signaling pathway by the strong agonist TPA. Likewise, FDP-NV did not activate central pathway of apoptosis (Caspase-3) in either FDP-NV concentration (FIG. 17). Taken together, the data suggest that the physical presence of the FDP-NV-BSA appears to subject HUVEC to some stress conditions at the Cmax, but not at the nadir exposure level. However, the ability of HUVEC to fully migrate (see FIG. 14) even at the highest FDP-NV exposure, suggesting disparities of functional sensitivity to the intra-cellular particle load.

HepG-2 cells generally appear to be resilient across some tests as compared to the HUVEC. In some cases, adverse effects of NDP on HepG-2 cell migration at the same exposure levels used in the HUVEC ‘wound healing’ model (“scratch assay”) were observed. For example, exposure of HepG-2 cells to 50-100 μg/mL FND resulted in 25-50% inhibition of migration, which was further inhibited (90%) at 200 μg/mL over the same time frame (24 hrs). It is of interest to note that these exposure levels did not interfere with HepG-2 cell proliferation in line with data reported herein (FIG. 10). Differences between the two sets of data could represent variances in particle size (100 nm vs 800 nm) and physical properties of non-functionalized NDP of some existing systems vs. carboxy-functionalized FDP-NV used in the present disclosure.

In vitro studies showed either cell type was exposed to the particles over 24 hrs.; however, the pharmacokinetic data of some existing systems indicate that in vivo endothelial cells are exposed to Cmax levels of FDP-NV for no more than 15-30 minutes, as the fast clearance into the liver depletes blood levels to <10 μg/mL within 90 min after infusion of the particles. In this light, this indicated that FNDP-NV˜800 nm are observed in the cytosol of hepatocytes within 1-2 hrs. post cell exposure. However, within the relevant in vivo short exposure time the intracellular levels of particles are several folds lower compared to the prolonged (24hrs) in vitro fixed FDP-NV concentration. The resiliency of the HepG-2 line across all conditions of stress lends credibility to our in vivo observation of hepatocytes health following high dose FDP-NV-800 nm infusion to rats over 12 weeks follow up.

Taken together FDP-NV-800 nm had no adverse effect when infused in vivo to intact rats 18-20, nor were there any adverse consequences in cultured HepG-2 cells line across the 7 ‘stress tests’ these cells were subjected in vitro. In some cases, aberrant consequences related to immune-inflammatory cells or other cells/organs especially those with a high-capacity phagocytosis or priming effects that could exacerbate underlining pathological conditions could not be excluded. The results obtained in this study indicate that further development of FDP-NV˜800 nm for in vivo imaging, and as vehicle for the delivery of drugs and therapeutics may be warranted.

### Summary

The present example demonstrates the biocompatibility of FDP-NV˜700/800 nm with respect to endothelial (HUVEC) and hepatic (HepG-2) cells in vitro. This study appears distinct from existing systems in that it probes biocompatibility within the realm of the pharmacokinetics of the particles in vivo (in a rat model). It can be concluded that HUVEC are more sensitive than HepG-2 cells to FDP-NV˜800 nm accumulation; this observation has not been described for any negative response at top exposure level (Cmax). Considering the mild to moderate interferences in certain biochemical functions in HUVEC and considering the pharmacokinetics profile the particles display in vivo, it is plausible to predict limited aberrant consequences to the endothelium. The resilience of HepG-2 cells in each and all of the biochemical tests under the top dose of FDP-NV˜800 nm supports in vivo data on normal liver function in spite of the prolonged retention of the particles in this organ. Overall, the results obtained in this example indicate FDP-NV-800 nm may be useful for in vivo imaging, and/or as vehicle for the delivery of drugs and therapeutics.

### Example 3

This example describes characterization of fluorescent (nano)diamond particles (FDP) administered to mice.

### Material and Methods

### Materials

FDPs: FDP-NV-700/800 nm (FDP-NV) functionalized by surface enriched carboxylation moieties were purchased from ADAMAS Nanotechnologies Inc., (Raleigh NC). Doxorubicin used for FDP-NV coating was purchased from MedKoo Biosciences, (Morrisville, NC). FDP-NV-700/800 nm and FDP-DOX (doxorubicin-coated FDP-NV) were supplied as lyophilized (dry) powder deposited in sterile Eppendorf vials. FDP-NV-700/800 nm and FDP-DOX were characterized for diameter (Z-average) and surface charge (ζ-potential) using DLS (Malvern). Particles were sterilized at the manufacturer and suspended in sterile deionized water (DI) in sterile plastic Eppendorf tubes.

Liver cancer cells (Hep-3B-luc) and “nude” mice (NM): Hep-3B, liver cancer cells were used for a orthotopic liver tumor model in NM. Female nude mice (20-22 grams, 6-8 weeks) were purchased from Beijing Vital River Laboratory Animal Co., LTD.

### Methods

### Dispersion of Particles by Sonication Prior to Infusion

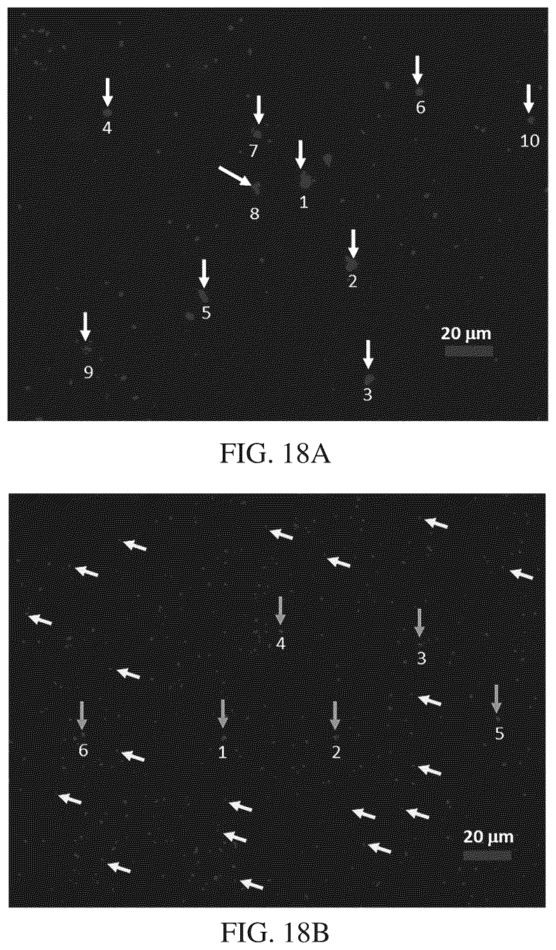

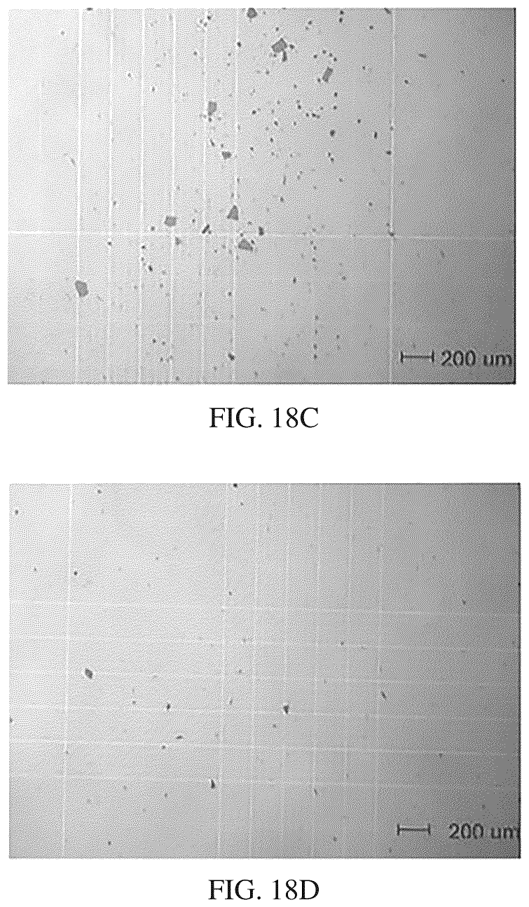

To facilitate dispersion of the particles prior to infusion of FDP-NV or FDP-DOX in vivo, BSA at 3% was added to PBS pH=7.4 and the suspension subjected to high vortex stir for 5 min. Immediately thereafter sonication in a water bath was performed for 10 minutes using ultrasonic cleaner machine Digital Pro+ (CO-Z, China) with power 80 W and frequency 40 kHz, maintaining water temperature in the range 20-25° C. An example of the process is provided in FIGS. 18A-18B.

### Cell Cultures

Hep-3B-luc were maintained in vitro in appropriate medium supplemented with 10% fetal bovine serum at 37° C. in with 5% CO₂ in air. The tumor cells were sub-cultured twice weekly, during the exponential growth phase and harvested for tumor inoculation at targeted inoculation at 3×10⁶.

### Orthotropic Tumor Model Development Using H3B Liver Cancer Cell Line

Hep3B-luc orthotopic liver cancer model was established by injecting 3×10⁶ of Hep3B-luc cells suspended in matrigel (1:1/w:w) into the liver of female Balb/c nude mice under proper anesthesia (see methods).

### Whole Body Bioluminescence of H3B-luc Cancer Cells

Inoculated mice were weighed and administered luciferin via tail vein at a dose of 150 mg/kg. Five minutes past luciferin injection the animals were lightly anesthetized with a gas mixture of oxygen and isoflurane. Upon proper anesthetic state mice were moved into the imaging chamber for bioluminescence measurements with an IVIS (Lumina II) imaging system. Whole body bioluminescence was recorded.

Bioluminescence of the tumor was also recorded ex vivo (after cardiac perfusion with saline) to minimize endogenous tissue interferences.

### Assignment to Groups

FDP-NV or FDP-DOX distribution in nude mice (task 1 A and B) was grouped randomly by body weight while FDP-DOX distribution study (Task 1C) was selected by using “Mouse Interventions Scoring System” (MISS, supplement 2)

### Ex Vivo Fluorescence Measurements

Distribution of FDP-NV-700/800 nm or FDP-DOX in whole animal (and selected organs ex vivo) were measured by IVIS Lumina III using setting Ex/Em at 580/710 nm, respectively, with auto exposure setting-time and “binning” set at 4. All ex vivo imaging of organs obtained from task 1A and B were performed on organs that were dissected on the 5^{th} day after perfusion with sterile saline. Organs harvested from studies conducted in task 1C were done 24 or 72 hrs. after FDP-DOX infusion (vide supra).

### Scanning Electron Microscopy (SEM)

SEM was used to confirm presence and appearance of particles in liver and tumor tissues isolated from FDP-DOX infused mice. Briefly, 800 micrograms/mouse of FDP-DOX were injected intravenously under proper anesthesia. 5 days thereafter, mice were euthanized and subjected to whole body perfusion via cardia puncture followed by dissection of the liver-tumor unit. The dissected tumor specimens were immersed in 70% ethanol for fixation and sliced at of 2-3 mm width. Slices were imaged using an environmental SEM (Quanta 450FEG, FEI Co., ThermoFisher Scientific) operated in low vacuum mode at 0.3-0.4 Torr of water evaporation and 7-10 keV of acceleration voltage. The scanning electron imaging was performed by Professor D. Dikin, PhD College of Mechanical Engineering, Nano Instrumentation Center facility at Temple University Philadelphia, PA, USA.

### Animal Anesthesia and Euthanasia

Mice were sacrificed by exsanguination while under deep (5% isoflurane) anesthesia, perfused with 10 mL sterile saline via cardiac puncture to flush out minimize residual blood in the organs'vasculature. Organs were preserved in either 70% ethanol or formalin buffered PBS (pH=7.4).

### Alfa-Fetoprotein (AFP) Immunohistochemistry

Alpha feto-protein (AFT) was used as a biomarker to differentiate Hep-3B-luc human liver tumor cells from mice hepatocytes. Five micron sections were prepared and stained by DAPI or HE method. Fluorescence imaging scanning utilized DAPI channel for cell nuclei.

### Infusion of Particles'Suspensions to Mice

Suspensions of various doses of FDP-NV or FDP-DOX were injected into the mice via the tail vein under proper anesthesia. Intra-tumor injection of a single dose of FDP-DOX suspension was done via the same injection line by which H3B cancer cells were injected to the left liver lobe.

### Data Analysis and Statistics

Data are presented as mean±1 SD. Statistical analyses were done by ANOVA (where appropriate) using SigmaPlot software (SigmaPlot® 12 SPSS, Systat Software Inc., San Jose CA, USA). Statistical significance was established at value of P<0.05. Plots were prepared using SigmaPlot Software. For non-linear regression dynamic fitting plot, the standard four parameters logistic curve was drafted using SigmaPlot software.

### Results

### Characterization of FDP-NV and FDP-DOX Particles by DLS

FDP-DOX-35/40 displayed a modest, 5.7% increase in diameter and a large change in surface charge, relative to FDP-NV, which increased to +41 mV charge. The change in surface charge resulted from association of the DOX with the FDP surface.

### Experimental Design

Preferential deposition of FDP-DOX in livers harboring clusters of cancer cells, as well as associated pharmacokinetics and distribution of the FDP-DOX within liver and tumor clusters were studied to identify a maximum tolerated dose (MTD), a parameter that secures maximum deposition of the therapeutic agent while avoiding ‘spill-over’ into the systemic circulation. Spill-over into systematic circulation can increase risk of systemic adverse effects. Finally, the dynamics of H3B tumor development were characterized within the liver with respect to time, the extent of tumor spread in situ, and the outgrowth of the tumor into the abdominal space and organs.

Five pilot studies were designed, each aimed at validating a discrete assumption.

Task 1A validated that FDP-NV and FDP-DOX were preferentially deposited in the livers of mice following systemic (IV) administration.

Task 1B tested the maximum retained dose (MRD) of FDP-DOX in NM livers, and liver function tests (LFTs) and complete blood counts (CBC) were performed. A high MRD is associated with a low spill-over of FDP-DOX, and can be associated with reduced non-specific, adverse effects.

Task 1C tested whether an FDP-DOX infused IV could be used to administer FDP-DOX to liver and tumor tissues 5 weeks after inoculation of mice with orthotopic cancer cells. FDP-DOX in tumor outgrowth into the abdominal cavity, along with preservation of LFT and hematological variables, was measured. Task 1C further tested for the presence of free DOX in liver and tumor tissue, which could serve as evidence of desorption of the DOX from the FDP.

### Experimental Results

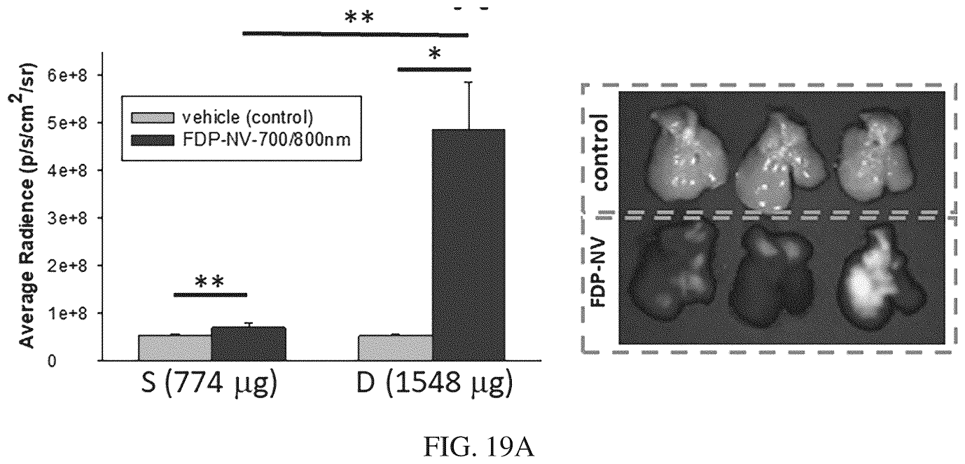

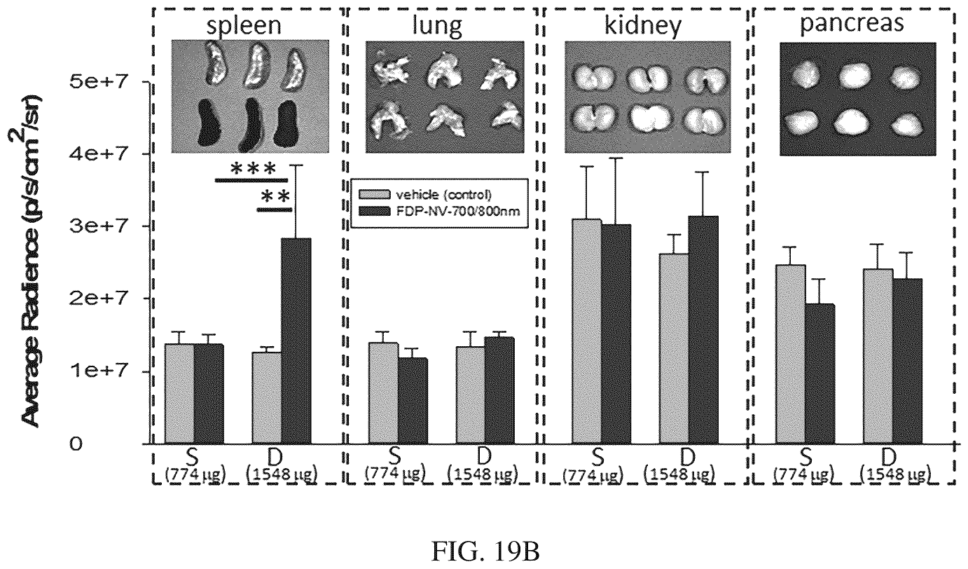

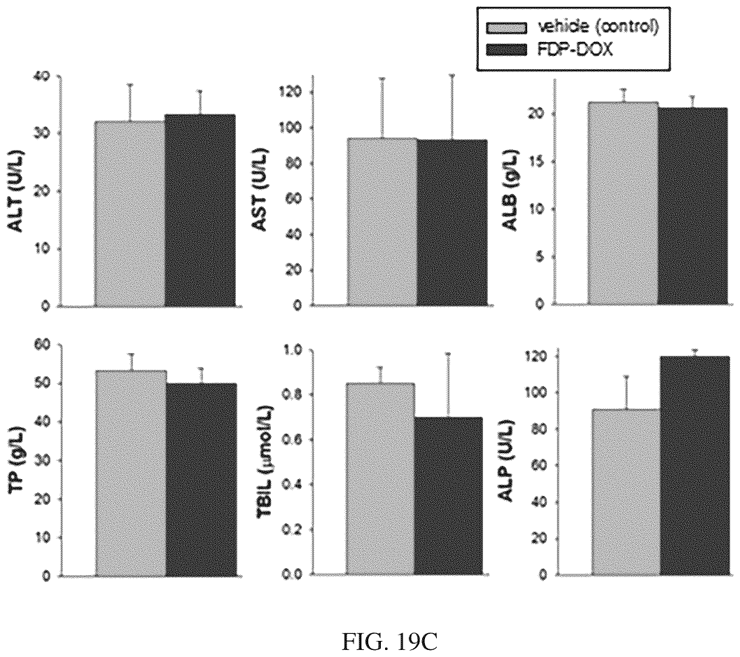

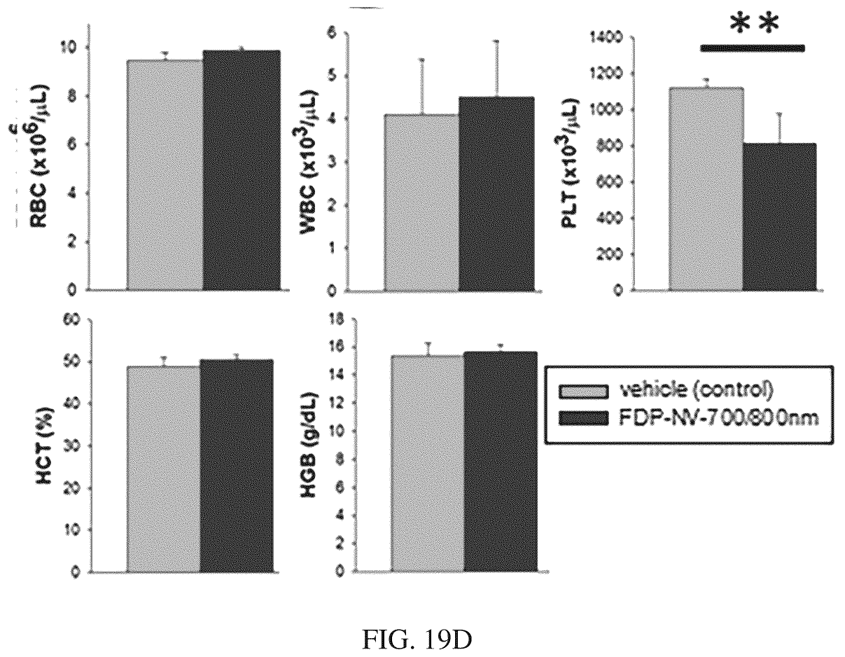

Task 1A: FIG. 19A demonstrates accumulation of FDP-NV (naïve) particles in mice livers, based on near-IR (NIR) radiance measurements. A single dose, 744 microgram, dose of FDP-NV (marked as “S”) had a slightly higher signal than the signal from vehicle (control) mice. However, doubling the dose to 1548 micrograms (marked as “D”) resulted in a more than tenfold increase in NIR signal, as shown. FIG. 19B illustrates minor (twofold) accumulation of particles in the spleen when administering a double dose (D), relative to a single dose(S). However, no significant accumulation was observed in the lung, kidney or pancreas tissue, as shown. NIR fluorescence images of the livers are displayed in FIG. 19A and fluorescence images of the rest of the tested organs (lung, kidney, pancreas, spleen) are shown in FIG. 19B. FIGS. 19C-19D present LFT and CBC variables on the 6^{th} day after FDP-NV injections, demonstrating no statistically significant change from normal levels.

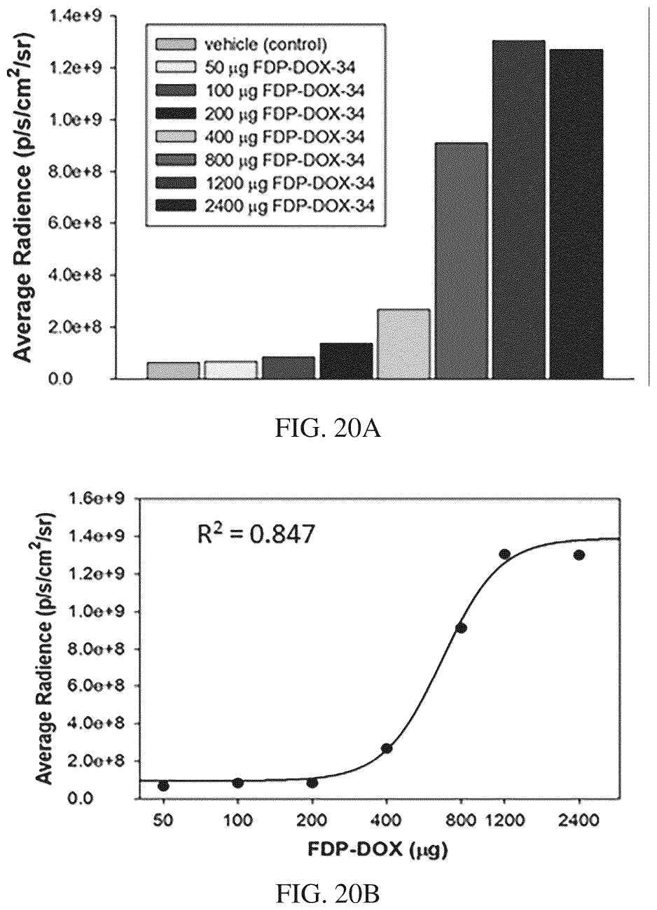

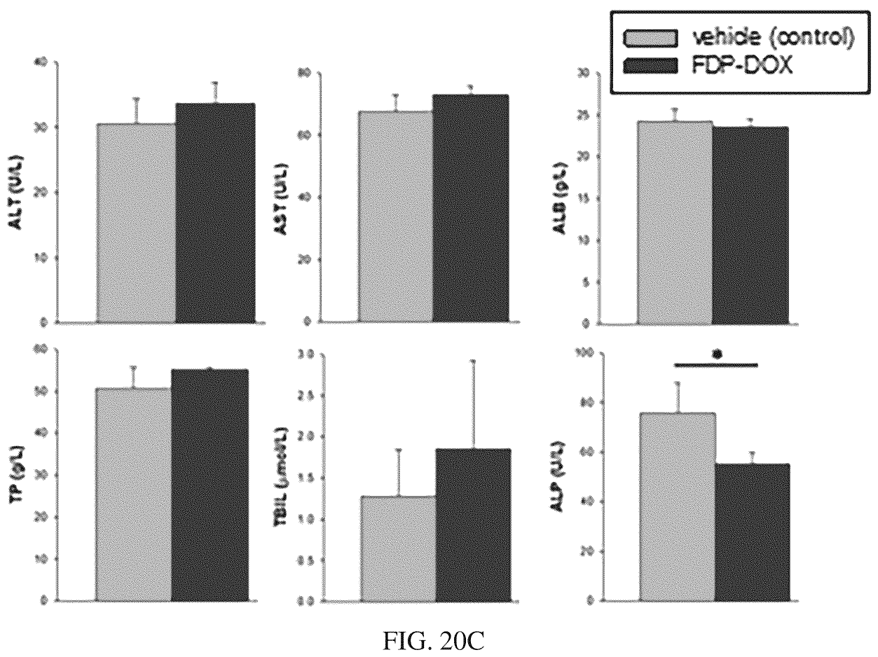

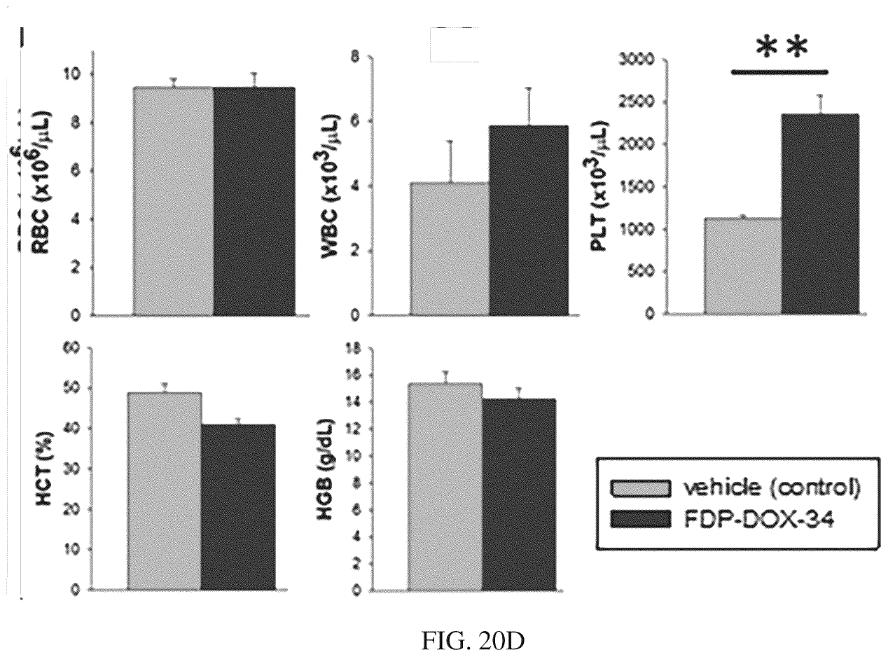

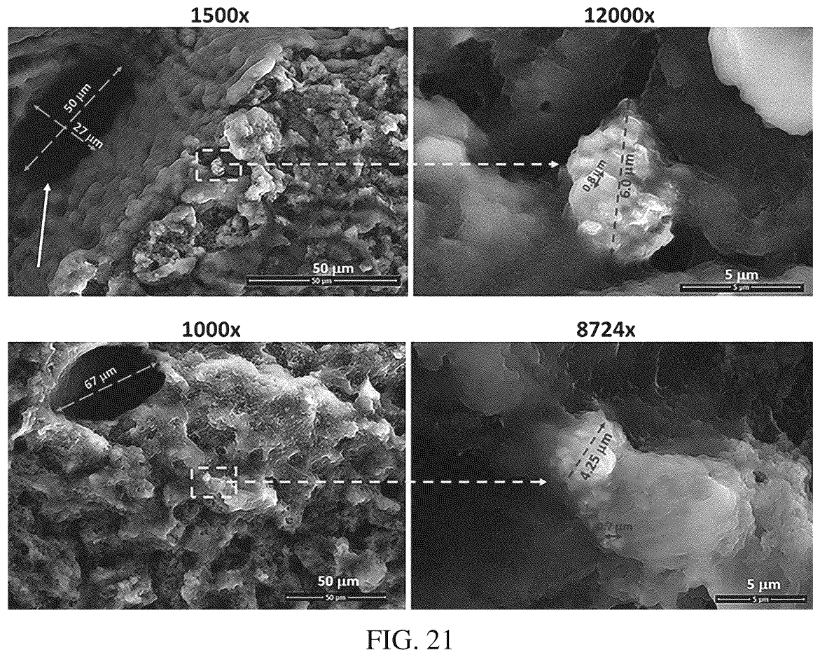

Task 1B: FIGS. 20A-20D present an intensive dose-response study using FDP-DOX to explore the liver maximum (saturable) capacity to retain the DOX coated particles. FIG. 20A presents ex vivo NIR fluorescence (IVIS) following each dose displayed as mean NIR+/−1 standard deviation (SD) for a varying number of mice studied at each of the respective dose level. FIG. 20B illustrates a logistic display of accumulation of FDP-DOX in the liver reaching. The top two dose levels, 1,200 micrograms/mouse and 2400 micrograms/mouse, saturated the liver tissue, identifying the likely MRD for FDP-DOX. FIGS. 20C-20D present the results of LFT and CBC variables, respectively, for the 1200 micrograms/mouse dose. No LFT or CBC variable differed significantly, relative to the vehicle (control) except for modest reduction of ALP (part C, Alkaline Phosphatase).

FIG. 21 presents scanning electron microscopy (SEM) of slices (3-5 um) obtained from liver isolated from the 1200 micrograms/mouse dose. Agglomerated clusters of particles were observed in spaces likely to represent sinusoids. Magnifications are shown for each micrograph. At the highest magnifications (8724× and 12,000×), individual particles are noted within clusters of 4-6 um. Individual particles within the clusters (see dashed arrows) appeared to have the same diameter measured by DLS.

### Task 1C

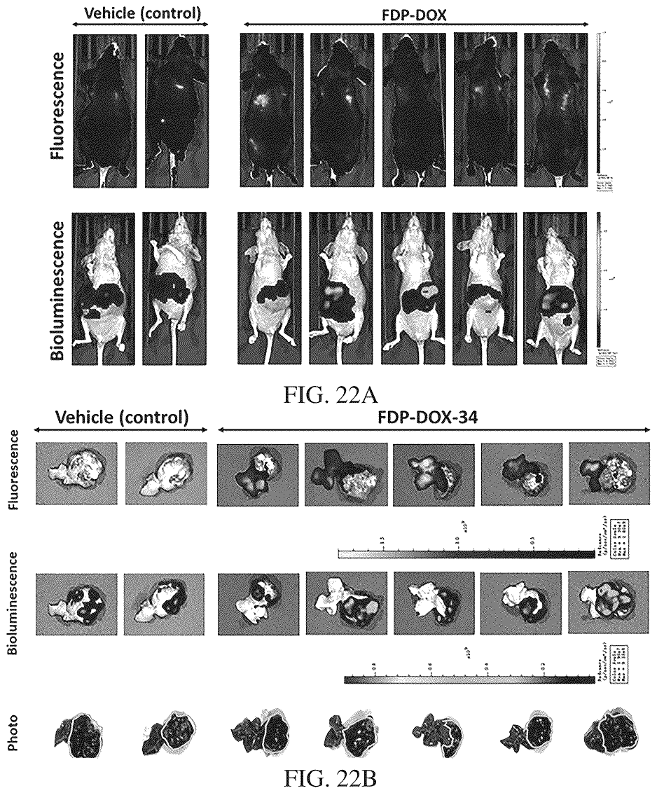

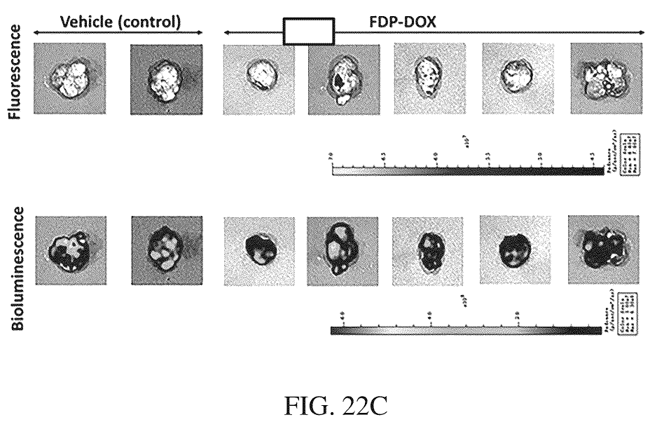

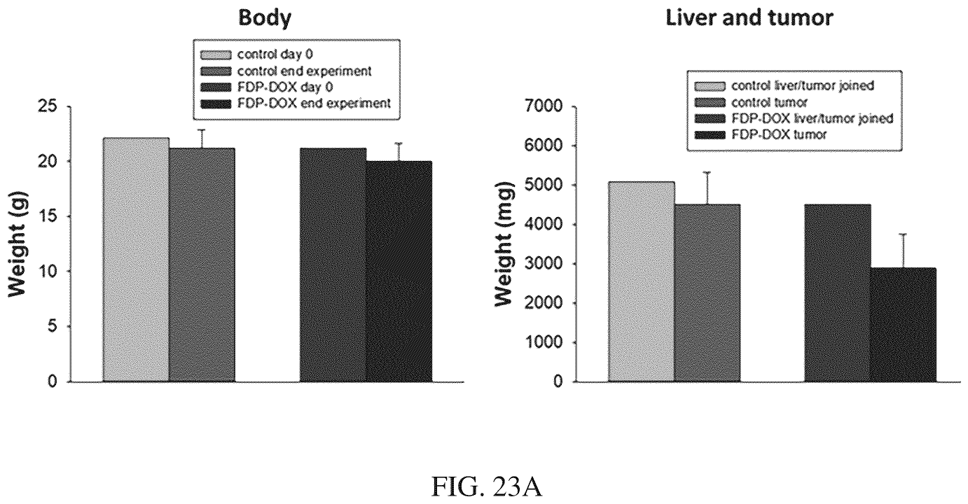

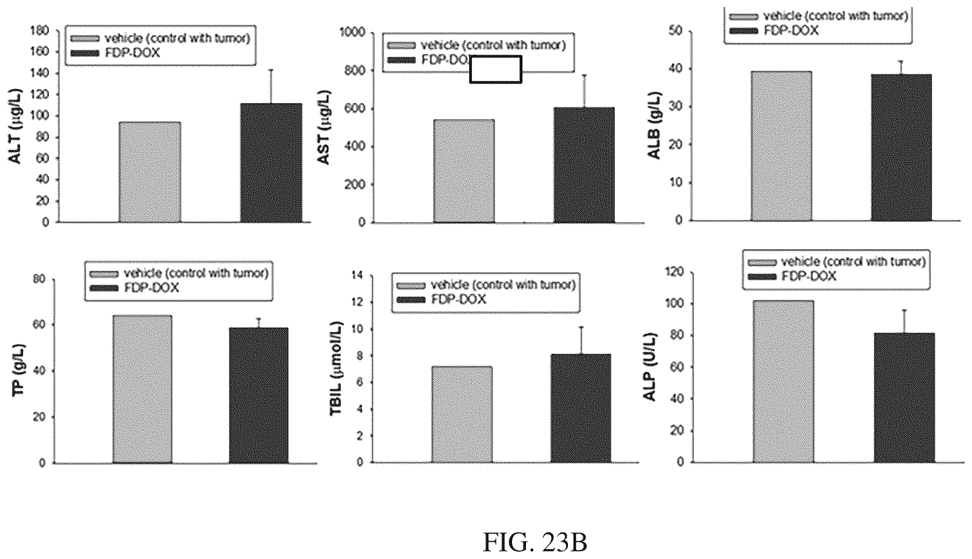

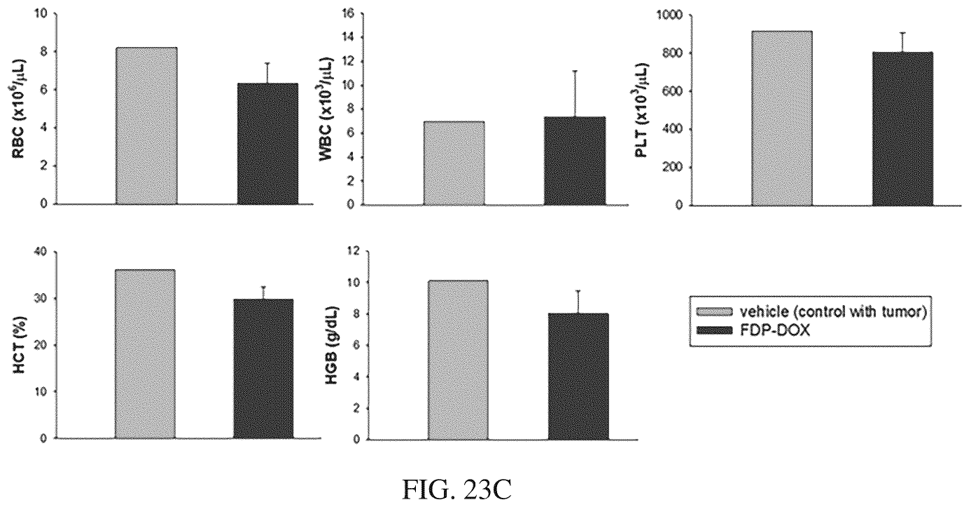

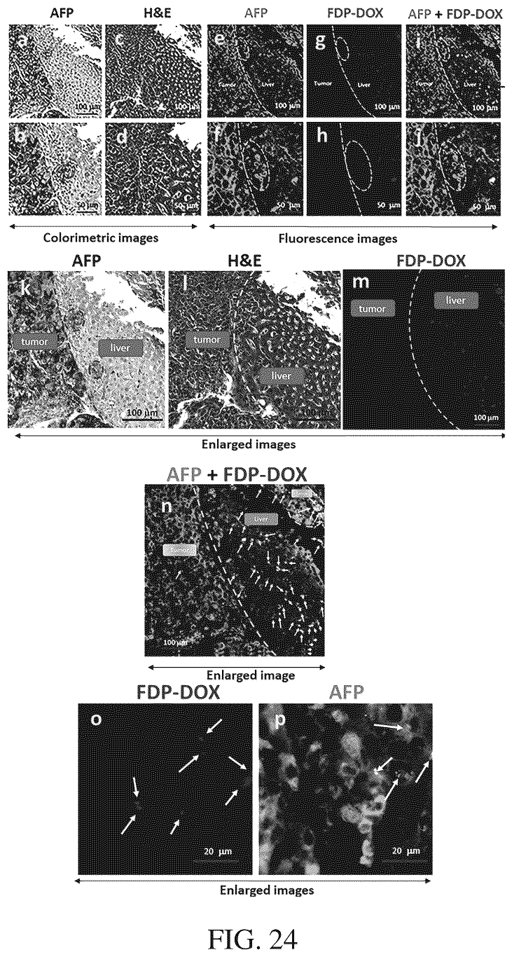

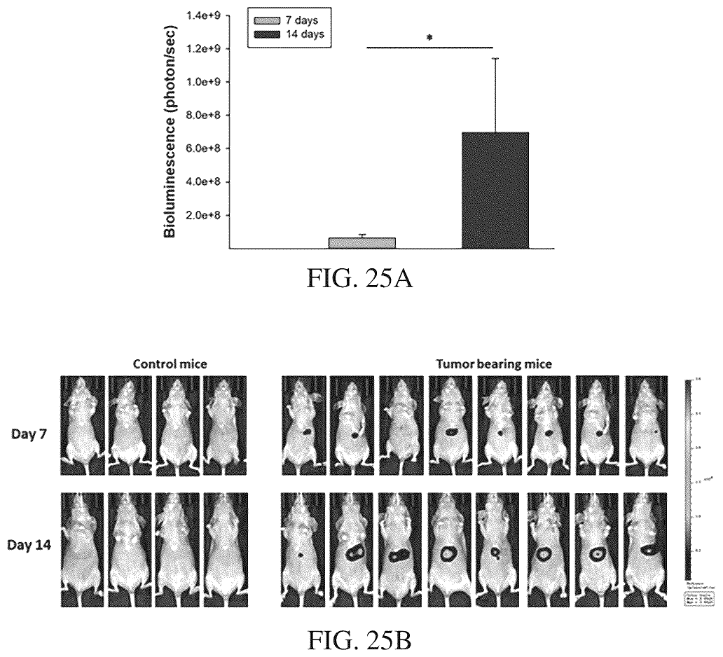

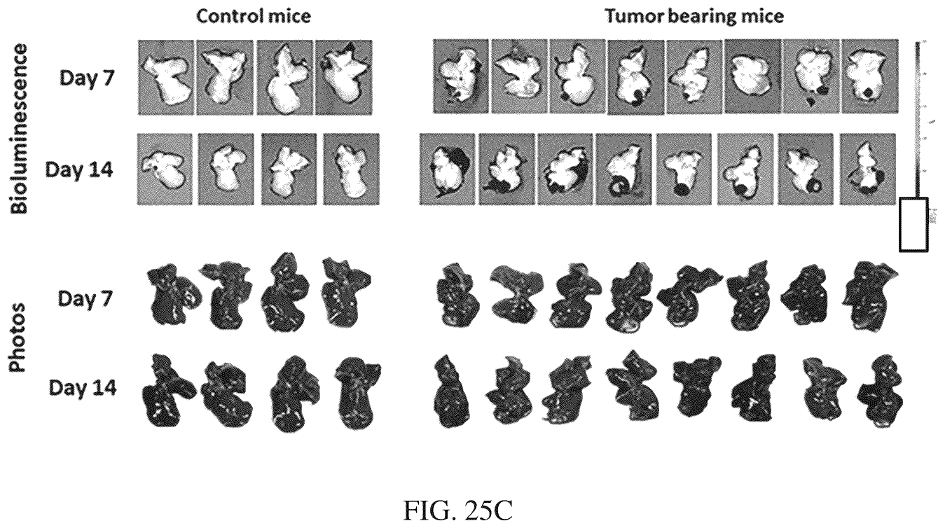

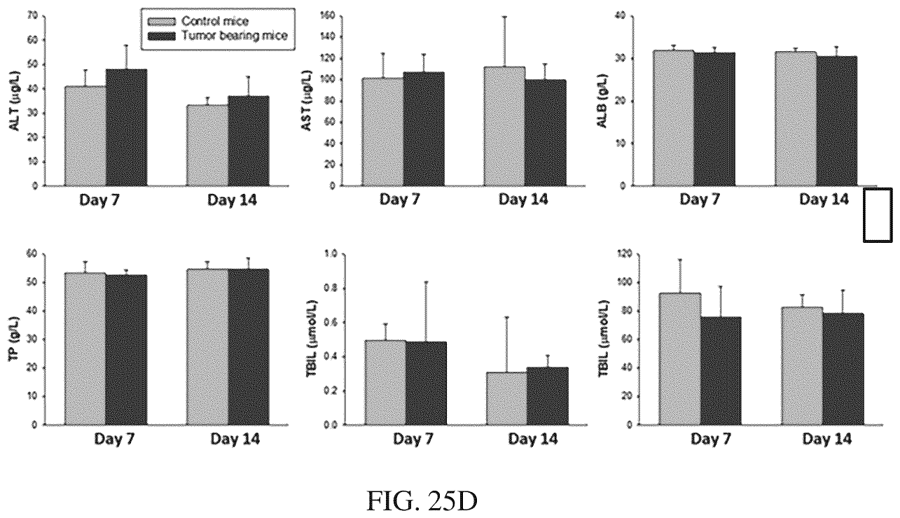

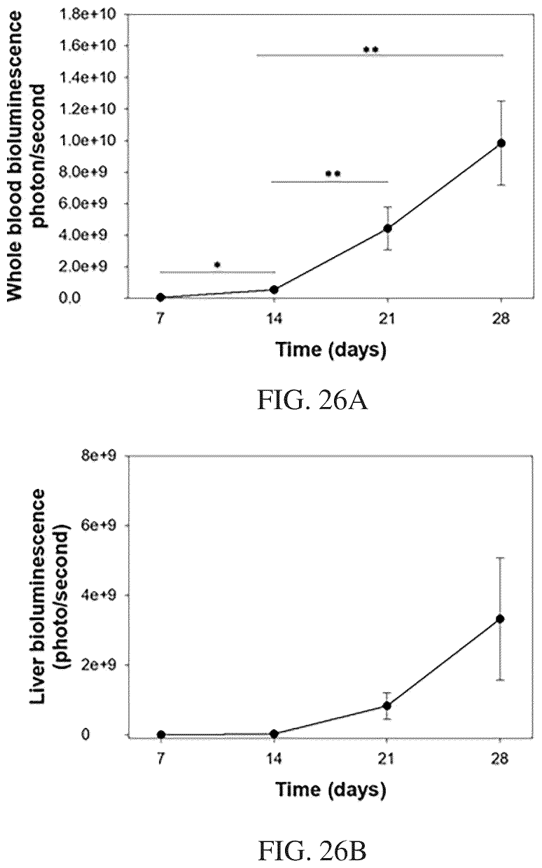

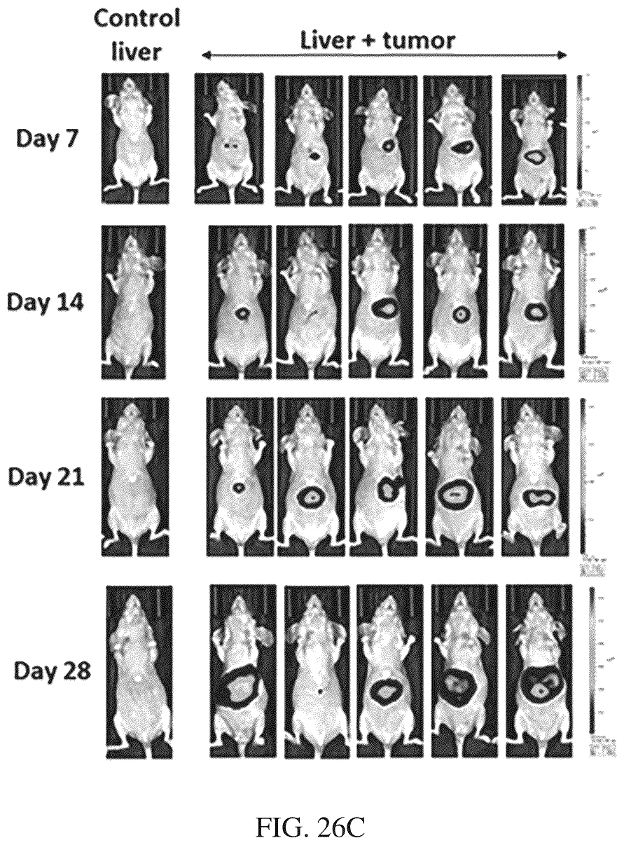

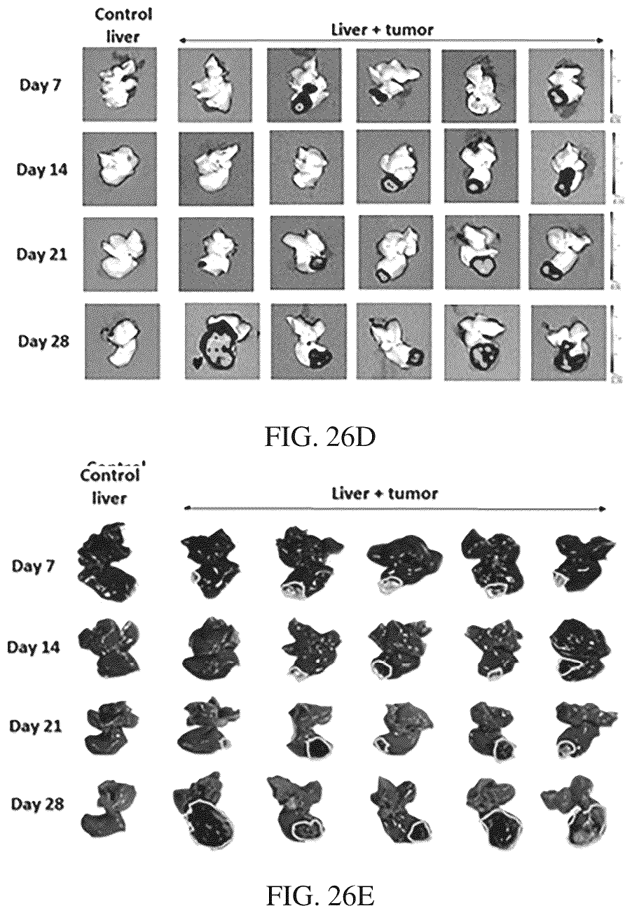

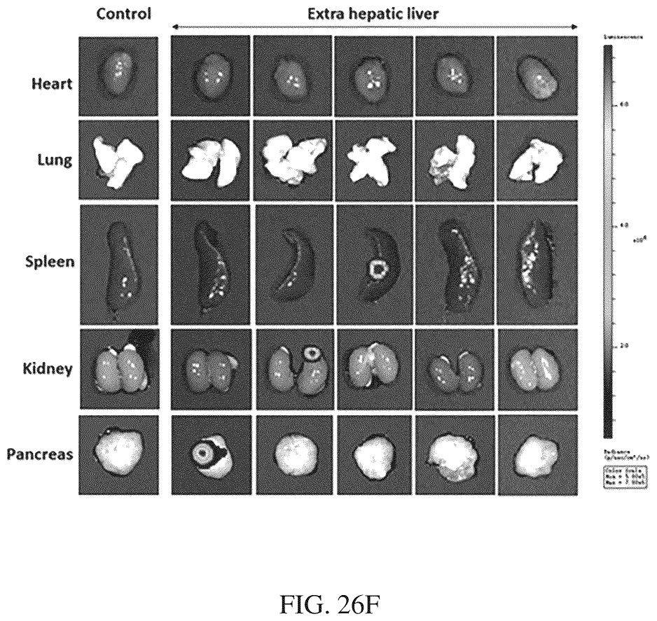

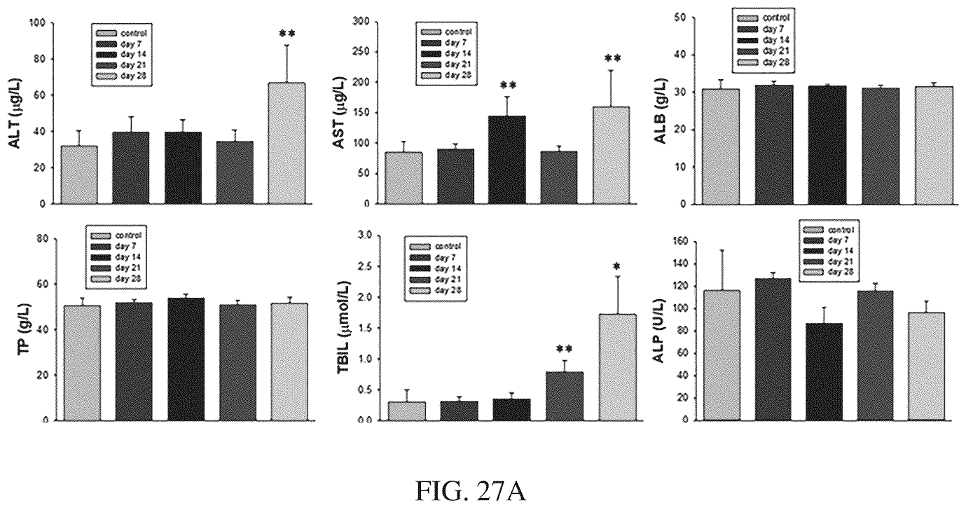

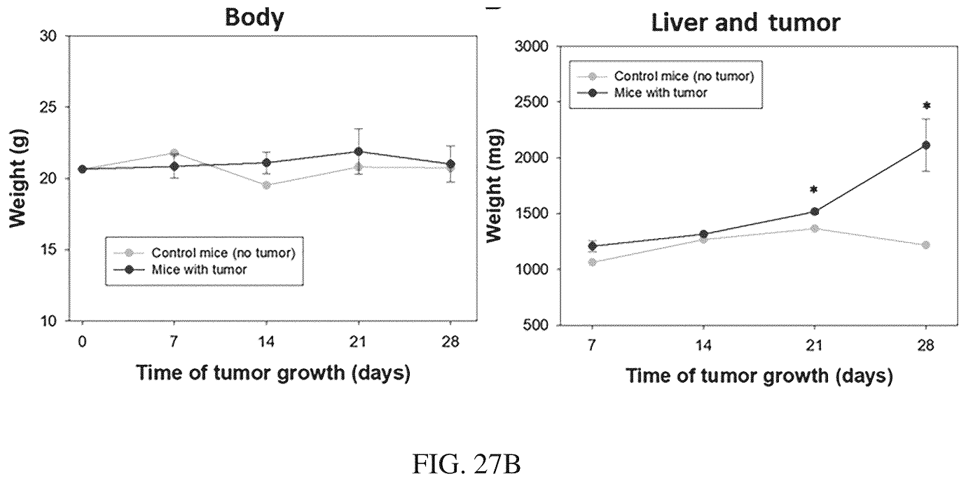

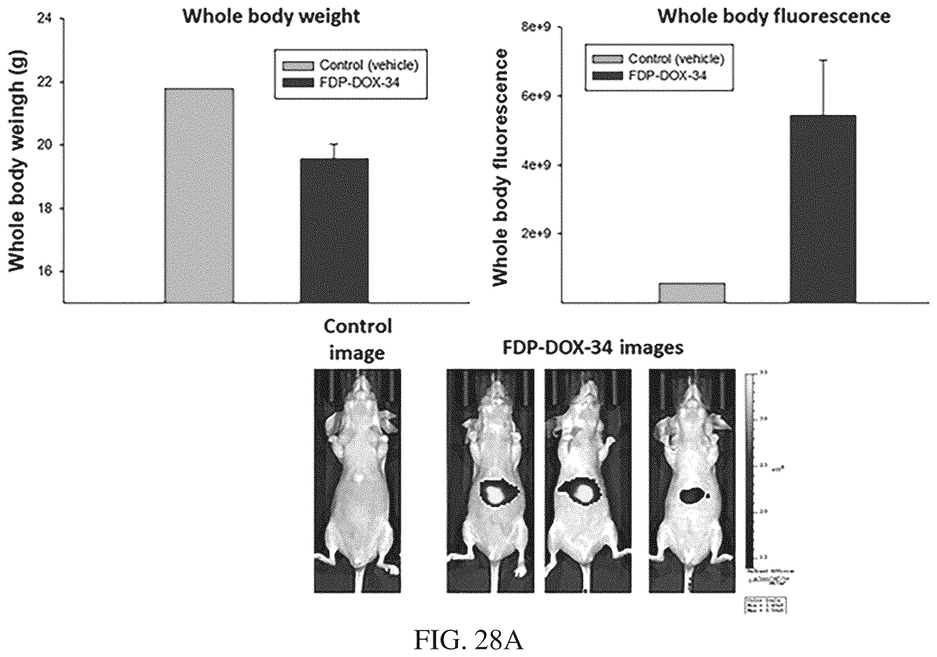

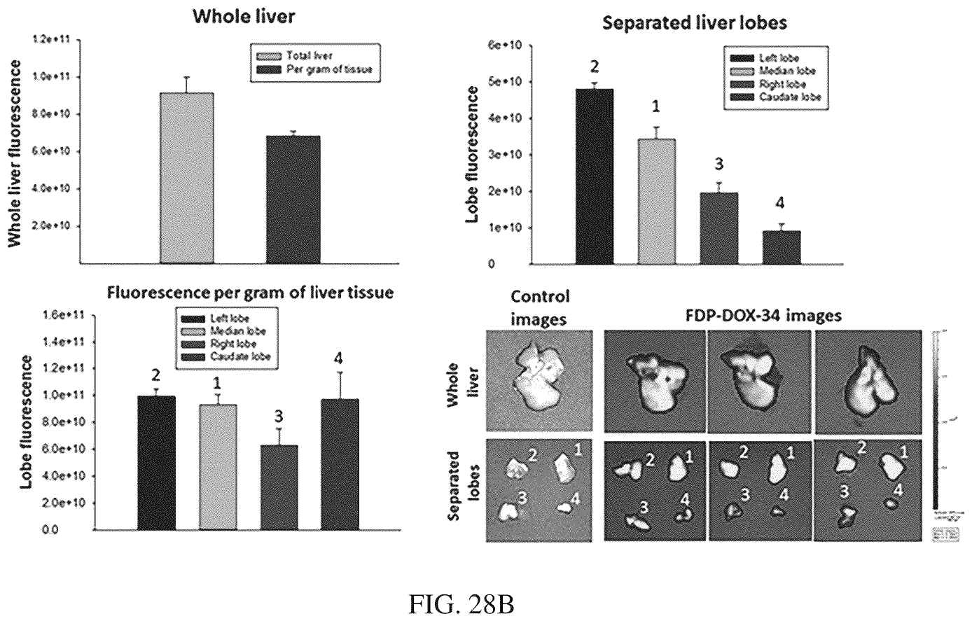

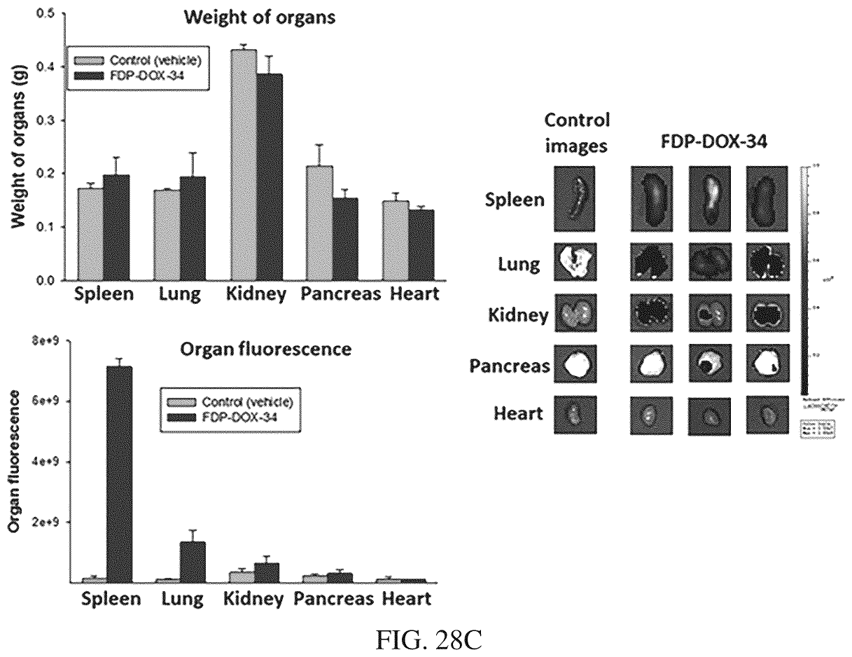

FIG. 22A, upper row, presents fluorescence obtained from whole body images of 5 FDP-DOX treated mice (maximum dose) and 2 vehicle controls. The images indicate endogenous NIR fluorescence sources (upper row) in vehicle control and augmented emission in FDP-DOX treated mice. Specific tumor-associated emission obtained by Luciferin bioluminescence images generated in intact mice illustrate (lower row) tumor cells in the abdominal cavity. FIG. 22B presents images obtained ex vivo, where NIR emitted from FDP-DOX treated mice is clearly projected (upper row) but not in the vehicle controls. Notably, tumors external to the liver were similar in size between the treated and non-treated mice (visible in middle and lower row) as a result of the limited time particles reside in the mice (mice were euthanized 24 hrs. after FDP-DOX injection). Tumors external to the liver had minimal FDP-DOX deposition, even within well-developed tumors. This contrasts with the greater FDP-DOX deposition for tumors internal to the liver and suggests a disparity of access of FDP-DOX to the liver, relative to the tumor out-growth external to the liver. Overall, the intense study of Task 1C indicates that acute treatment with ‘top dose’ of FDP-DOX did not manifest acute adverse effects as compared to vehicle control mice. FIG. 23A presents the weight of vehicle control mice bodies and livers, relative to the weight of the bodies and livers of mice treated with FDP-DOX. FIGS. 23B-23C present the results of LFT and CBC variables, respectively, for the mice. No significant difference was observed between the weights or LFT and CBC variables of the mice.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition for detecting, monitoring and/or treatment of cancer, the composition comprising:
a plurality of diamond particles; and
an anti-cancer agent bound to at least a portion of the plurality of diamond particles,
wherein the plurality of diamond particles have an average particle diameter of greater than or equal to 500 nanometers and less than or equal to 2 microns,
wherein the plurality of diamond particles are sized and configured for prolonged residence internal to an organ of a subject.

2. A composition as in claim 1, wherein the organ is the liver or pancreas of the subject.

3. A composition as in claim 1, wherein the composition is capable of prolonged detection, monitoring and/or treatment of cancer in the organ of the subject.

4. A composition as in claim 1, wherein the anti-cancer agent is present in the composition in an amount of 1 mg per 250 mg of fluorescent diamond particles.

5. A composition as in claim 1, wherein the anti-cancer agent is selected from the group consisting of cyclophosphamide, methotrexate, 5-fluorouracil, vinorelbine, doxorubicin, docetaxel, bleomycin, vinblastine, dacarbazine, mustine, vincristine, procarbazine, prednisolone, etoposide, cisplatin, epirubicin, capecitabine, methotrexate, vincristine, folinic acid, oxaliplatin, gemcitabine, ifosfamide, and etoposide.

6. A composition as in claim 1, wherein prolonged residence is greater than or equal to 1 day, 3 days, 5 days, 7 days, 14 days, or 21 days.

7. A composition as in claim 1, wherein the plurality of diamond particles comprise (auto)fluorescent diamond particles having an emission wavelength of greater than or equal to 650 nm and less than or equal to 1000 nm.

8. An article as in claim 1, wherein the plurality of diamond particles are configured to aggregate within the organ of the subject.

9. An article as in claim 1, wherein the plurality of diamond particles are configured to be captured by the organ of the subject.

10. An article as in claim 1, wherein an aggregate of diamond particles has an average diameter of greater than or equal to 1 micron and less than or equal to 100 microns.

11. An article as in claim 1, wherein the plurality of diamond particles have a characteristic near infrared emission.

12. A method of treating a disease, comprising:
administering intravenously, to a subject, a plurality of diamond particles and an anti-cancer agent bound to at least a portion of the diamond particles, wherein the plurality of diamond particles have an average particle diameter of greater than or equal to 500 nanometers and less than or equal to 2 microns, wherein the plurality of diamond particles is configured for prolonged residence internal to an organ of a subject.

13. A method as in claim 12, wherein the plurality of diamond particles are present in the liver of the subject.
